(19) United States
(12) Patent Application Publication
(10) Pub. No.: US 2007/0236213 A1
(43) Pub. Date: Oct. 11, 2007

(54) TELEMETRY METHOD AND APPARATUS USING MAGNETICALLY-DRIVEN MEMS RESONANT STRUCTURE

(76) Inventors: Bradley E. Paden, Goleta, CA (US); Brian Norling, Santa Barbara, CA (US); Josiah E. Verkaik, Goleta, CA (US)

Correspondence Address: Dennis R. Haszko;Patent Law Office of D.R.Haszko, 499 MOSHER HILL ROAD, FARMINGTON, ME, 04938-5405, US

(21) Appl. No.: 11/278,138

(22) Filed: Mar. 30, 2006

(51) Int. Cl. A61B 5/05; G01R 33/12

(52) U.S. Cl. 324/228; 600/409

(57) ABSTRACT

A telemetry method and apparatus using pressure sensing elements remotely located from associated pick-up, and processing units for the sensing and monitoring of pressure within an environment. This includes remote pressure sensing apparatus incorporating a magnetically-driven resonator being hermetically-sealed within an encapsulating shell or diaphragm and associated new method of sensing pressure. The resonant structure of the magnetically-driven resonator is suitable for measuring quantities convertible to changes in mechanical stress or mass. The resonant structure can be integrated into pressure sensors, adsorbed mass sensors, strain sensors, and the like. The apparatus and method provide information by utilizing, or listening for, the residence frequency of the oscillating resonator. The resonant structure listening frequencies of greatest interest are those at the mechanical structure's fundamental or harmonic resonant frequency. The apparatus is operable within a wide range of environments for remote one-time, random, periodic, or continuous/on-going monitoring of a particular fluid environment. Applications include biomedical applications such as measuring intraocular pressure, blood pressure, and intracranial pressure sensing.

### CROSS REFERENCE TO RELATED APPLICATIONS

This application claims does not claim any benefit of priority.

### STATEMENT OF FEDERALLY SPONSORED RESEARCH OR DEVELOPMENT

This application is not currently the subject of any U.S. Government sponsored research or development.

### FIELD OF THE INVENTION

The present invention relates generally to an apparatus including a resonant structure suitable for measuring quantities convertible to mechanical stress or mass in the resonant structure and a related method. More particularly, the present invention relates to an apparatus and method including a magnetically-driven resonant sensor suitable for wireless physiological parameter measurement and telemetry within a living body.

### BACKGROUND OF THE INVENTION

Within the field of biomedical devices, the measurement of physiological parameters within a living body presents unique problems. Such problems and related known solutions can be found, for example, in the treatment of glaucoma which is a highly significant concern to the medical community. Glaucoma is a serious disease that can cause optic nerve damage and blindness. There are a number of causes of glaucoma, but increased intraocular pressure is the primary mechanism. Because of the large number of persons suffering from glaucoma combined with the seriousness of the disease and the need for early detection and optimized drug treatment, it is desirable to obtain frequent measurements of eye pressure. Moreover, eye pressure can vary throughout the day such that clinical diagnosis, based on infrequent testing, is often delayed. It is therefore desirable to obtain fast and accurate pressure monitoring.

The surgical placement of a sensor in the eye (i.e., intraocular) may be advisable in patients with glaucoma or in patients with a risk of glaucoma if they are undergoing eye surgery for another reason. In particular, patients receiving an intraocular lens (IOL) can be fitted with pressure sensors attached to the IOL with little additional health risk or cost. Also, glaucoma patients who need to adjust their drug dosage according to eye pressure would benefit from such a device.

There have been a number of past devices directed at the measurement of intraocular pressure. A prevalent technique exists that employs contacting the cornea of the eye using a tonometer. The cornea is topically anesthetized and brought into contact with the smooth, flat surface of the tonometer probe. The amount of pressure required to flatten a specified area of the cornea is used to compute the intraocular pressure. While this method is cost effective, it suffers from a number of significant drawbacks. For example, a trained clinician is required for the measurement so that frequent monitoring is not possible. Further, the mechanical properties of the cornea can affect the measurement. Still further, the tonometer needs to be maintained in clean and sterile conditions.

It has elsewhere previously been proposed to provide a technique for continuously monitoring eye pressure involving an inductor-capacitor (LC) resonant circuit wherein the resonant frequency was sensitive to eye pressure. However, such devices were not sufficiently compact and reliable for clinical use in humans, and lacked a method of implantation and attachment. Moreover, LC resonant sensors fail to provide a sufficiently sharp resonance to allow for rapid and simple external sensing of frequency and hence pressure. Such sensors may exhibit a quality factor (Q) in the range of 30. The Q factor is a measure of the “quality” of a resonant device or system. Resonant systems respond to frequencies close to their natural frequency much more strongly than they respond to other frequencies. The Q factor indicates the amount of resistance to resonance in a system. Systems with a high Q factor resonate with greater amplitude (at the resonant frequency) than systems with a low Q factor. Damping decreases the Q factor. Modifications to known LC resonators using planar microelectromechanical systems (MEMS) manufacturing technologies have been attempted. However, the problems of low Q associated with resistive losses in the coil and other conductors remained due to sensitivity of such system to the relative position of the sensor and the inductive pick-up coil.

While still other pressure sensors derived from a mechanical resonator have been suggested that could be small enough for implantation in the eye and still have a high Q, such sensors often use light to drive a photo-diode that electrostatically attracts a resonant beam or otherwise provides an optical excitation system delivering the requisite high light intensities to the sensor. The relatively high intensity light requirements may interfere with the patient's vision or may otherwise not likely be suitable for use near the human eye.

There also exist a number of LC resonant pressure sensors with wireless communication. Such schemes rely on magnetic coupling between an inductor coil associated with the implanted device and a separate, external “readout” coil. For example, one known mechanism of wireless communication is that of the LC tank resonator. In such a device, a series-parallel connection of a capacitor and inductor has a specific resonant frequency that can be detected from the impedance of the circuit. If one element of the inductor-capacitor pair varies with some physical parameter (e.g., pressure), while the other element remains at a known value, the physical parameter may be determined from the resonant frequency. Such devices using LC resonant circuits have been proposed in various forms for many applications such as hydrocephalus applications, implantable devices for measuring blood pressure, and implantable lens for monitoring intraocular pressure.

Implantable wireless sensors have also existed within the treatment of cardiovascular diseases such as chronic heart failure (CHF). CHF can be greatly improved through continuous and/or intermittent monitoring of various pressures and/or flows in the heart and associated vasculature. While applications for wireless sensors located in a stent have been suggested, no solution exists to the difficulty in fabricating a pressure sensor with telemetry means sufficiently small enough for incorporation into a stent.

In nearly all of the aforementioned cases, the disclosed devices require a complex electromechanical assembly with many dissimilar materials. This typically results in significant temperature and aging-induced drift over time. Such assemblies may also be too large for many desirable applications—e.g., including intraocular pressure monitoring and/or pediatric applications. Finally, complex assembly processes make such devices prohibitively expensive to manufacture for widespread use. Such manufacturing complexity only increases with alternative process that form microfabricated sensors which have recently been proposed as an alternative to conventionally fabricated devices.

There have also been attempts to offer telemetry sensors using magneto-mechanical pressure sensors of the magnetostrictive type. Magnetostriction is a property of a ferromagnetic material that changes volume when subjected to a magnetic field. When biased by a non-alternating magnetic field, magnetostrictive material stores energy via mechanical strain. This storage affects the Young's modulus, E, of the material. Such magnetostrictive materials can be caused to resonate in an alternating magnetic field. Resonant frequency can be designed by varying the geometry of the material, one or more mechanical properties of the magnetostrictive material, and strength of the biasing non-alternating magnetic field. These types of sensors have a high magnetic permeability element. The high magnetic permeability element is placed adjacent to an element of higher magnetic coercivity. The high magnetic permeability element being adjacent to the element of higher magnetic coercivity resonates when interrogated by an alternating electromagnetic field due to nonlinear magnetic properties. The high magnetic permeability element adjacent to the element of higher magnetic coercivity generates harmonics of the interrogating frequency that are detected by a receiving coil. Such sensors can have a thin strip of magnetostrictive ferromagnetic material placed adjacent to a magnetic element of higher coercivity (often referred to as “a magnetically hard element”).

As suggested above, the non-alternating magnetic bias placed on the magnetostrictive material causes a mechanical strain in the magnetostrictive material that in turn affects a resonant frequency of the magnetostrictive material. The resonance of the magnetostrictive material can be detected electromagnetically. While magneto-mechanical pressure sensors have advantages such as high operating reliability and low manufacturing cost over previous electromagnetic markers of high sensitivity, there are known problems associated with such a pressure sensor. The magnetostrictive response is temperature sensitive, primarily due to a dependence on Young's modulus. Consequently, such magnetostrictive pressure sensors often require independent temperature correction that involves the use of additional temperature and measurement devices that add size and preclude construction as a single monolithic structure or adaptation to a micro-miniature size suitable for monitoring physiological parameters.

Further known types of mechanical resonant sensors have been used for many years to achieve high accuracy measurements. Vibrating transducers have been used in accelerometers, pressure transducers, mass flow sensors, temperature and humidity sensors, air density sensors, and scales. Such sensors operate on the principle that the natural frequency of vibration (i.e., resonant frequency of an oscillating beam or other member) is a function of the induced strain along the member. One of the primary advantages of resonant sensors is that the resonant frequency depends only on the geometrical and mechanical properties of the oscillating beam, and is virtually independent of electrical properties. As a result, precise values (e.g., resistance and capacitance) of drive and sense electrodes are not critical. A possible disadvantage is that any parasitic coupling between the drive and sense electrodes may diminish accuracy of the resonant gauge. Furthermore, in a conventional capacitive drive arrangement, the force between the oscillating beam and drive electrode is quadratic, resulting in an unwanted frequency pulling effect. While crystalline quartz piezoresistors have been satisfactorily employed in resonant gauge applications, their size limits their practical utility.

Recently, other known types of pressure sensing devices have been fabricated from semiconductor material—e.g., silicon. In general, pressure sensing devices of this type are realized adopting so-called “silicon micromachining” technologies. Such technologies provide two or three-dimensional semiconductor structures with mechanical properties that can be well defined during design, despite their extremely small size (down to a few tens of microns). Accordingly, such semiconductor structures are capable of measuring and/or transducing a mechanical quantity (for example the pressure of a fluid) with high accuracy, while maintaining the advantages, in terms of repeatability and reliability that are typical of integrated circuits. Such pressure sensing devices made of semiconductor materials of the so-called “resonant-type” pressure sensing devices have become widespread in the industrial field. Ultra miniaturized sensors for minimally invasive use have become important tools in heart surgery and medical diagnoses during the last ten years. Typically, optical or piezoresistive principles have been employed in such sensors. Although these devices have considerable advantages, such as, for example, high accuracy and stability of measurement even for very wide measurement ranges (up to several hundred bars), such known sensors suffer from some drawbacks. In particular, calibration is fairly complicated and manufacture is not an easy task, producing fairly high rejection rates of the finished products. Accordingly, there is much unresolved need for new types of sensors and other means and methods of making ultra miniaturized sensors in an efficient and economic way.

There are also known related devices pertaining to magnetically driven cantilevers for use in atomic force microscopes and imaging processes involving magnetic force microscopy. Still further, there are known related devices pertaining to micro-compasses with magnetically coupled resonant structures. However, such cantilevers and micro-compasses fail to provide a solution in measuring other quantities convertible to measuring changes in mechanical stress (i.e., pressure and force).

In view of the above and other limitations on the prior art, it is apparent that there exists a need for an improved sensor system. It is, therefore, desirable to provide a wireless MEMS system utilizing a magnetically-driven resonator for use in physiological parameter measurement capable of overcoming the limitations of the prior art and optimized for signal fidelity, transmission distance, and manufacturability. It is further desirable to provide a magnetically-driven MEMS resonator adapted for wireless physiological parameter measurement including resonant structure attached to magnetic material used to drive structure resonance.

### SUMMARY OF THE INVENTION

In general, the present invention relates to telemetry using sensing elements remotely located from associated pick-up, and processing units for the sensing and monitoring of pressure within an environment. More particularly, the invention relates to a unique remote pressure sensing apparatus that incorporates a magnetically-driven resonator (whether hermetically-sealed within an encapsulating shell or diaphragm) and associated new method of sensing pressure. The resonant structure is suitable for measuring quantities convertible to changes in mechanical stress or mass. This structure can, for example, be integrated into pressure sensors, adsorbed mass sensors, and strain sensors. The present invention includes a magnetically-coupled MEMS resonator that provides improvements over known devices including increased reliability and ease-of-use.

The pressure sensing apparatus and method(s) in accordance with the present invention provide information by utilizing, or listening for, the residence frequency of the oscillating resonator. The resonant structure listening frequencies of greatest interest are those at the mechanical structure's fundamental or harmonic resonant frequency. The pressure sensing apparatus of the invention can operate within a wide range of environments for remote one-time, random, periodic, or continuous/on-going monitoring of a particular fluid environment.

Any of a number of applications for the present apparatus and method is contemplated including, without limitation, biomedical applications (whether in vivo or in vitro). The resonant structure in accordance with the present invention is driven and sensed remotely, allowing use in applications where connection by way of wires is impractical or not otherwise feasible. In particular, the present apparatus and method is suitable for biomedical applications including measuring intraocular pressure in patients with glaucoma or patients at risk for contracting glaucoma and having intraocular lenses (IOL's). While this specific application relating to glaucoma and measurement of intraocular pressure is discussed in detail, it should be understood that such specific example is merely illustrative of the present invention and other biomedical applications with the same limitations as the intraocular environment may equally benefit from the present invention such as, but not limited to, blood pressure sensing and intracranial pressure sensing. Moreover, the present invention may be useful in applications pertaining to rotating machinery, not limited to biomedical applications, as another specialized application where wires are often impractical.

Energy is transmitted to the resonant structure magnetically and the motion of the structure is detected magnetically, optically, or acoustically. Magnetic drive is particularly useful because of the ability to provide high forces with the magnetic drive coils separated by a sizable distance. The sensing apparatus of the present invention is useful to measure intraocular pressure, but can be applied to any sensing application where the sensed variable can affect a change in stress or mass in a mechanical resonator so that its frequency is altered. In the case of intraocular pressure, structure motion may be detected magnetically or optically.

In one embodiment of the invention, a magnetic material is mounted on a torsional resonator. Pressure is converted to tension in the resonator beams so that its frequency is correlated to pressure. The torsional resonator is excited by a nearby current carrying coil and the same coil can be used for sensing the resonant frequency. The coil is connected to a grid dip meter or other circuit to enable the measurement of the resonance. The sensor may be hermitically sealed in a miniature capsule and attached to an IOL implanted in the eye. Alternatively, it can be attached directly to the iris. A variation on this embodiment replaces the permanent magnet with a soft magnetic material such as nickel-iron, cobalt-iron or other alloy that can be easily attached or formed onto the resonator. During use, soft magnetic material is magnetized with a permanent magnet external to the eye. The resonator is excited with a coil as mentioned above.

An advantage of the present invention is the high quality factor (Q) that is attainable with mechanical resonant structures relative to LC resonant circuits and the improved reliability and ease-of-use of a sensor based on a high-Q resonator. Further, magnetic couplings allow for communication with the sensor through biological tissues. The resonant structure includes a magnetic material and is adapted to vibrate in response to a time-varying magnetic field. The apparatus also includes a receiver to measure a plurality of successive values magnetic field emission of the vibrating structure taken over an operating range of successive interrogation frequencies to identify a resonant frequency value for said sensor.

Another aspect of the present invention is to provide a pressure sensing apparatus for operative arrangement within an environment that incorporates a resonant structure with at least one magnetically-driven resonant beam that will vibrate in response to a time-varying magnetic field (whether radiated continuously over an interval of time or transmitted as a pulse). The resonant beam may be enclosed within a hermetically-sealed diaphragm, at least one side of the diaphragm having a flexible membrane to which the resonant structure is coupled. The pressure sensing apparatus also includes a receiver unit capable of picking up emissions (whether electromagnetic or acoustic) from the sensor. Preferably, the receiver (a) measures a plurality of successive values of coil resistance corresponding to the frequency of the sensor taken over an operating range of successive interrogation frequencies to identify a resonant frequency value for the sensor, or (b) detects a transitory time-response of resonance intensity of the sensor due to a time-varying magnetic field pulse to identify a resonant frequency value thereof. In the latter case, the detection can be done after a threshold amplitude value for the transitory time-response of residence intensity has been observed; then a Fourier transform can be performed on the transitory time-response of the emission to convert the detected time-response information into the frequency domain.

It is an aspect of the present invention to provide a sensing apparatus for measuring quantities convertible from changes in physical observations, the apparatus including: a resonant structure responsive to the changes in the physical observations, the resonant structure including a magnetized element; an electromagnetic coil operationally coupled to the magnetized element, the electromagnetic coil being an excitation coil magnetically coupled to the magnetized element to excite a resonance of the resonant structure; and, a signal processor for processing movement of the resonant structure, the signal processor correlating the movement with regard to the changes in the physical observations so as to produce sensed data. The resonant structure includes: a substrate locatable in an environment to be monitored, a flexible diaphragm hermetically sealed to the substrate and in communication with the environment to be monitored, a sealed chamber encompassed by the substrate and the at least one flexible diaphragm, and a resonant beam connected to the magnetized element, the resonant beam suspended within the sealed chamber and mechanically coupled to the flexible diaphragm, wherein the magnetized element oscillates the resonant beam in response to an electromagnetic signal generated by the signal processor and formed by the electromagnetic coil.

It is another aspect of the present invention to provide a method of sensing physical observations within an environment, the method including: operatively arranging a resonant structure in the environment and in proximity to a direct current bias field, the resonant structure including a magnetized element and being responsive to changes in the physical observations; applying a magnetic field by way of an electromagnetic coil operationally coupled to the magnetized element; measuring a plurality of successive values for magnetic resonance intensity of the resonant structure with a signal processor operating over a range of successive interrogation frequencies to identify a resonant frequency value of the resonant structure; and using the resonant frequency value to identify sensed data correlating to the physical observation of the environment.

Many advantages exist by providing the flexible new pressure sensing apparatus of the invention and associated new method of sensing pressure of an environment using a sensor with at least one magnetically-driven resonant structure. Such advantages include, but are not limited to, the following:

(a) Sensitivity—The method provides a means for achieving high sensitivity and high-Q resonance frequency.

(b) Simplicity—Resonance frequency is easily measure, and the small devices can be manufactured in arrays having desired acoustic response characteristics.

(c) Speed—Much faster response time (tens of microseconds) than conventional acoustic detectors (tens of milliseconds) due to extremely small size and large Q value.

(d) Variable Sensitivity—The sensitivity can be controlled by the geometry of the microbeam(s) and the coating thereon. This can be made very broadband, narrow band, low pass, or high pass.

(e) Size—Current state-of-the-art in micro-manufacturing technologies suggest that a mechanical structure could be mounted on a monolithic MEMS structure.

(b) Low power consumption—The power requirements are estimated to be in sub-milliwatt range for individual sensors.

(d) Low cost—No exotic or expensive materials or components are needed for sensor fabrication. Electronics for operation and control are of conventional design, and are relatively simple and inexpensive.

(e) The invention can be used for one-time (whether disposable), periodic, or random operation, or used for continuous on-going monitoring of pressure changes in a wide variety of environments; Sensor materials and size can be chosen to make one-time, disposable use economically feasible.

(f) Versatility—The invention can be used for operation within a wide range of testing environments such as biomedical applications (whether in vivo or in vitro).

(g) Simplicity of use—The new sensor structure can be installed/positioned and removed with relative ease and without substantial disruption of a test sample or environment.

(h) Structural design flexibility—the resonant structure may be formed into many different shapes and may be fabricated as a micro-circuit for use where space is limited and/or the tiny sensor must be positioned further into the interior of a sample or environment being tested/monitored.

(i) Several sensors may be positioned, each at a different location within a large test environment, to monitor pressure of the different locations, simultaneously or sequentially.

(j) Several sensor elements may be incorporated into an array to provide a package of sensing information about an environment, including pressure and temperature changes.

(k) Receiving unit design flexibility—One unit may be built with the capacity to receive acoustic emissions (elastic nonelectromagnetic waves that can have a frequency up into the gigahertz (GHz) range) as well as frequency of the resonant structure, or separate acoustic wave and electromagnetic wave receiving units may be used.

Other advantages and benefits may be possible, and it is not necessary to achieve all or any of these benefits or advantages in order to practice the invention. Therefore, nothing in the forgoing description of the possible or exemplary advantages and benefits can or should be taken as limiting.

### BRIEF DESCRIPTION OF THE DRAWINGS

The novel features of the present invention, which are considered as characteristic for the invention, are set forth in this disclosure, but not with particularity according to limiting claims. The invention itself, however, both as to organization and methods of operation, together with further objects and advantages thereof, may best be understood by reference to the following description, taken in conjunction with the accompanying drawings in which:

FIGS. 1a and 1b show top and side views, respectively, of a basic resonator structure with attached permanent magnet.

FIG. 2a shows a coil and resonator structure.

FIGS. 2b-2d show three of the many modes of vibration of the resonator illustrated in FIG. 2a.

FIGS. 3a and 3b show an embodiment of the resonator structure with a soft magnetic material.

FIGS. 4a and 4b show a dynamically balanced embodiment with minimal base motion.

FIG. 5 shows an alternative embodiment with two magnets on the same beam.

FIG. 6 shows an embodiment with additional flexures to allow alignment with a large external field;

FIG. 7 shows a resonant structure incorporated into a pressure sensor.

FIG. 8 shows an embodiment of an adsorption-type chemical sensor.

FIG. 9 shows a pressure sensor incorporated into an intraocular lens.

FIGS. 10a and 10b show coil placements outside of an eye.

FIG. 11 shows transmit and receive signals to/from the coil.

FIG. 12 illustrates the signal structure.

FIG. 13 shows the signal processor of the present invention.

FIGS. 14a and 14b show software functions for the receiving signal.

FIG. 15a shows a perspective view of an alternative embodiment of a resonant structure in accordance with another embodiment of the invention.

FIG. 15b shows a top view of the resonant structure of FIG. 15a that illustrates the resonant structure.

FIGS. 16a through 16c illustrate three possible shapes in which resonant structures may be fabricated.

FIG. 17a illustrates a layer of fabrication of a pressure sensor in accordance with another embodiment of the invention.

FIG. 17b is a top view illustration of the top layer of the resonant structure of FIG. 17a shown after being patterned.

FIG. 17c is a cross-sectional view of the resonant structure of FIG. 17b taken across the axis F-F, after the top layer of the resonant structure has been patterned.

FIG. 17d is a top view illustration, similar to that of FIGS. 15a and 15b, wherein a solid magnet has been bonded to the central bridge portion of the resonant bridge.

FIG. 17e is a cross-sectional view of the resonant structure of FIG. 17d across the axis F-F.

FIG. 17f is a top view of the patterned top level of the resonant structure of FIGS. 17d and 17e wherein a portion of a central layer of the resonant structure has been removed.

FIG. 17g is a cross-sectional view of the resonant structure of FIG. 17f across the axis F-F.

FIGS. 18a through 18c each illustrate vibration of the resonant structure of FIGS. 15a and 15b in three different modes of vibration.

FIG. 19a is a perspective view of a resonator of the double ended tuning fork (DETF) type.

FIG. 19b is a top view of an embodiment of a DETF resonator structure.

FIG. 20 shows a partial cutaway side view of a DETF resonator structure.

FIGS. 21a through 21c are illustrations indicative of the steps involved in producing mechanical resonators according to another embodiment of the present invention.

FIG. 22 is a cross-sectional representation of a pressure sensing resonator device embodying principals of the present invention.

FIG. 23 is a cross-section of an alternative embodiment of a sensor according to the present invention.

FIG. 24a is a cross-section of a second alternative embodiment of a sensor according to the present invention.

FIG. 24b is a cross-section detail of a suspension element according to an alternative embodiment of the present invention.

FIGS. 25a and 25b illustrate two embodiments of a microbeam structure according to the present invention.

FIGS. 26a and 26b illustrate the function of a sensor according to an alternative embodiment of the present invention.

FIG. 27 shows a circuit diagram of a data interpretation system of according to an embodiment of the present invention.

FIG. 28 shows a circuit diagram of an alternative embodiment of a data interpretation system according to the present invention.

### DETAILED DESCRIPTION

Generally, the present invention provides a method and apparatus including a magnetically-driven resonant structure suitable for measuring some change in a physical observation—e.g., sensing change in pressure, flow, etc. However, for purposes of illustration, the present invention is discussed in terms of a method and apparatus suitable for measuring intraocular pressure in patients having glaucoma or patients at risk of contracting the disease and having intraocular lenses (IOL's). As discussed earlier, previous devices fail to meet dimensional requirements, or they suffer from sensitivity limitations needed for wireless physiologic parameter measurement within a living body.

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways without straying from the intended scope of the present invention. Furthermore, unless otherwise indicated, the terms and expressions employed herein have been chosen for the purpose of describing the illustrative embodiments of the present invention for the convenience of the reader and are not for the purpose of limiting the invention. Further, it is understood that any one or more of the following-described embodiments, expressions of embodiments, examples, etc., can be combined with any one or more of the other following—described embodiments, expressions or embodiments, examples, etc.

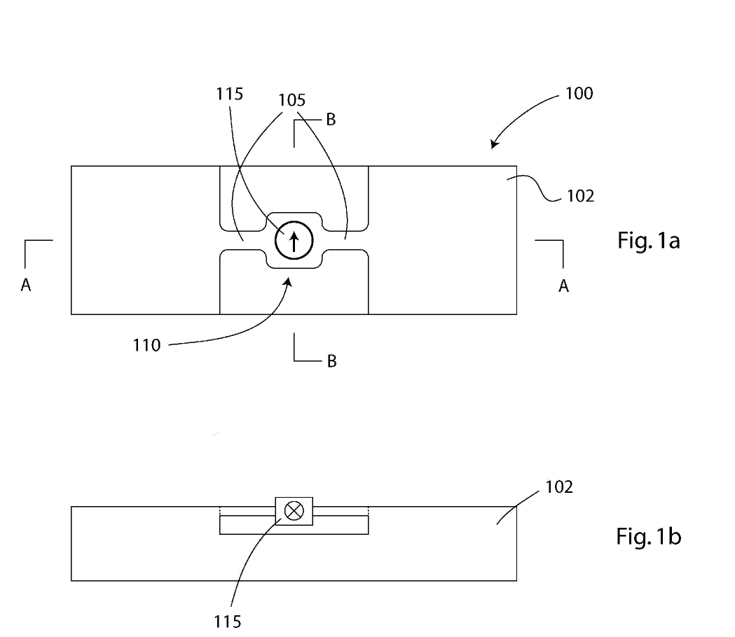

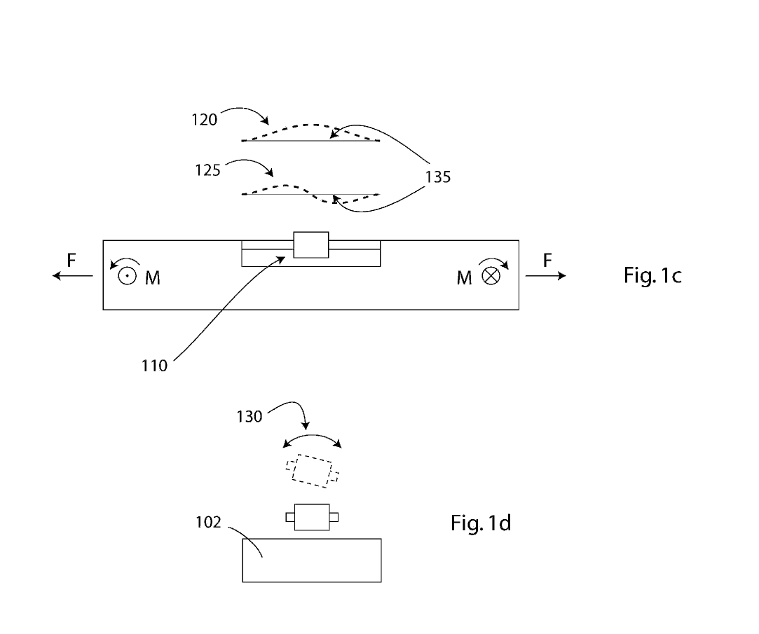

FIGS. 1a and 1b depict a simple embodiment of the invention. FIG. 1a is a top view and FIG. 1b is a section view along section A-A. In reference FIGS. 1a and 1b, a resonant structure 100 includes a body 102, elastic beams 105, a mass 110 and a magnetic material 115 mounted on the mass 110. The beam materials in particular are chosen such that they have relatively low damping and the mass can sustain a vibrational motion if excited. Typically, the body 102, elastic beams 105, and mass 110 are fabricated from the same elastic material. Suitable materials are single crystal silicon, polycrystalline silicon, titanium, brass or any other elastic material with low damping. As with many elastic systems, the resonant structure 100 can vibrate in a number of vibrational modes. As is done in the art, mode shapes and modal frequencies are associated with each vibrational mode.

Three such mode shapes are depicted in FIG. 1c. Mode shape 120 represents an up and down motion relative to the equilibrium position 135. At one extreme, the mass and elastic beams deflect upward to the mode shape 120. At the other extreme, the mass 110 and elastic beams 105 deflects downward to the mirror image of 120 relative to 135. Mode shape 125 represents a second vibrational motion of the mass 110 and beams 105 wherein the mass rotates back and forth about an axis pointing out of FIG. 1c. Another mode shape is associated with the motion 130 depicted in FIG. 1d.

In general, a resonant structure is any material body that vibrates at one or more frequencies. Examples include: stringed musical instruments, tuning forks, chimes, quartz crystals in watches, and microelectromechanical systems (MEMS) with vibrating components such as MEMS vibrational gyros. In the case of a guitar, the frequencies of vibrations include those of the strings, including their harmonic motions.

An advantage of the embodiment shown in FIGS. 1a through 1c is simplicity. However, vibrations of the beams and mass are accompanied by vibrations of the body. Consequently, if the body is brought into contact with a support structure, vibrational energy is drawn from the resonant structure and the vibration decays away more quickly than in resonant structures where the support locations vibrate little or not at all. The rate of decay of a vibration is captured in the notion of a quality factor (Q) by those practicing the art of vibration analysis. Higher quality factors reflect more sustained vibrations and can be as high as 1,000,000 in some single crystal resonant structure made from quartz or silicon.

In reference to FIG. 1c, forces F and/or moments M transmit stresses to the resonator structure and tension to the beams 105 in particular. Such stresses change the modal frequencies. Such a system is an example of a frequency variable resonator dependent on force. Force is an example of a sensed quantity and the embodiment of FIG. 1c can function as a force sensor. Mode shape 130 has a modal frequency that is relatively independent of beam tension when the beams are cylindrical rods. Hence, the cross section and choice of mode must be optimized to obtain the best sensitivity. This is easily done with commercial finite-element analysis (FEA) software packages such as COSMOS™ or ANSYS™. Because many sensed quantities such as pressure, strain, acceleration, and chemical concentration can be converted to stress in the resonant structure, the embodiment of FIGS. 1a through 1c can be incorporated into various sensors. Further, the rotation of the body can cause amplitude variations and energy transfer between modes. Such a phenomenon can be used to design a vibrational gyro. In this later case, we say that the resonator is an amplitude variable resonator dependent on rotation. Rotation is another example of a sensed quantity.

The magnetic material 115 in FIG. 1a provides a mechanism to excite the vibration in the resonant structure by coupling externally applied magnetic fields to the magnet. Vibrations are particularly excited when the external magnetic field applies oscillatory forces and/or torques to the magnetic material at the modal frequencies. The coupling is further enhanced when the mode shape is such that the magnet translates or rotates significantly when the mode is excited. For example, mode shapes 120, 125, and 130 all rotate or translate the magnetic material. The magnetic material may be a magnetized hard magnetic material (i.e., a permanent magnet such as NdFeB, SmCo or Ferrite) or a soft magnetic material such as silicon-iron or cobalt-iron. When a soft magnetic material is used, it is preferable to magnetize the soft material with a DC field produced by an external permanent magnet or a DC current in a coil.

Relationships can be computed for the force/torque interactions between a magnetic material and a magnetic field, and the interaction between these forces/torques and the motion of a resonant structure. If geometries are simple, pencil and paper calculations can be used. More complex geometries can be analyzed with finite-element software. In this way, the entire system can be engineered and optimized prior to fabrication and testing.

Detection of motion in the invention of FIGS. 1a through 1c can be accomplished magnetically through, for example: the use of a pick-up coil; acoustically by detecting vibrations of the body directly or via a propagating medium; or optically by reflecting light (e.g., laser light) off a polished surface of the structure.

The fabrication of the embodiment of FIGS. 1a through 1c can be accomplished with a number of manufacturing methods. When the device is small, MEMS manufacturing methods using silicon are desired. These methods include photolithography, etching (e.g., anisotropic etching, isotropic etching, and deep reactive ion etching), and various bonding techniques. Unique to the present invention is the bonding of the magnetic material 115 to a resonant structure 100. If a hard (i.e., high coercivity) magnetic material such as NdFeB or SmCo is used, the magnetic material is preferably bonded to the remaining structure with epoxy, photoresist, or other suitable organic compound. Another method of attaching materials such as NdFeB is to electroplate the contacting surface with nickel and then gold. The gold can then be bonded to silicon thermally though eutectic bonding. Alternatively, if a soft magnetic material is attached, electroplating using methods developed for disk drive recording heads are preferred.

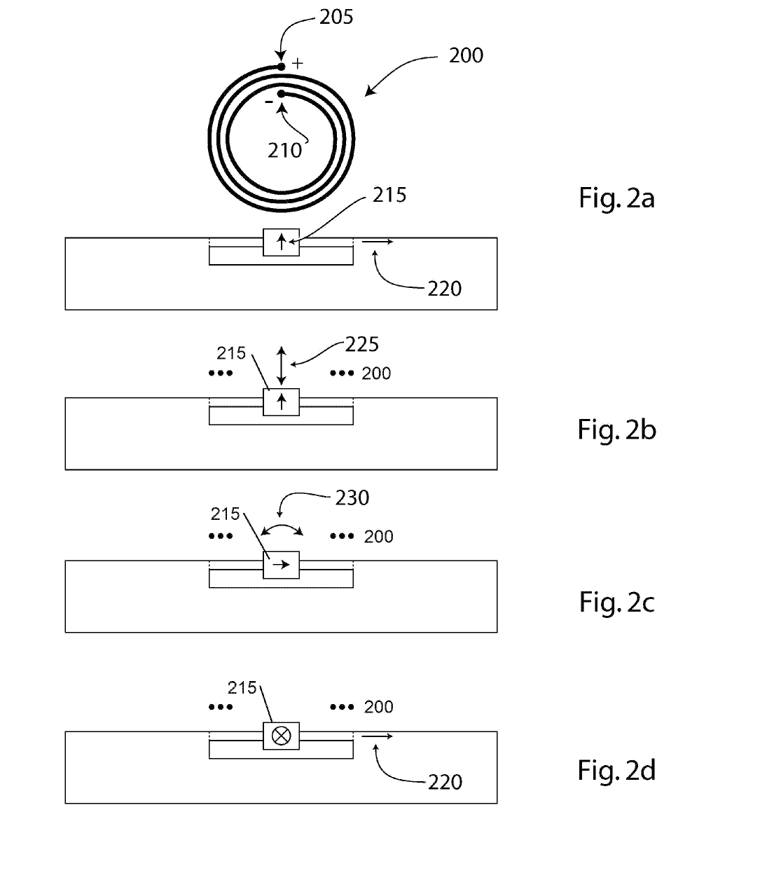

FIGS. 2a through 2d depict configurations for exciting and/or detecting vibrations when a permanent magnet (PM) is attached to the resonant structure in various orientations. The magnetization direction 215 is shown. FIG. 2a depicts a simple coil 200 with terminals 205 and 210 formed of insulated copper wire or another such suitable electrical conductor. To excite motion about the axis 220 in the resonant structure, electrical current is passed through such a coil 200 in order to produce a magnetic field. If the current waveform contains a frequency component at a resonant frequency, the corresponding vibrational mode can be excited. The orientation of the coil 200 relative to the PM direction of magnetization is important. For maximal torque application to the PM, the applied magnetic field should be perpendicular to the direction of PM magnetization. For maximal force application to the PM, the applied magnetic field gradient should be aligned with the direction of PM magnetization. In general, there will be a combination of torques and forces on the PM due to the combined effects of the magnetic field and the magnetic field gradient. Other angles differing from these can work well, but angles that differ from these by exactly 90 degrees produce no torque or force respectively.

The coil 200 can also sense rotary and linear motion of the PM as these motions generate a voltage across the coil terminals. Fortuitously, the relative position and orientation of the coil 200 and PM that maximize torque and force also maximize the voltage generated due to rotary and linear motion, respectively. While the application of a current while the sensing of voltage is one way to measure the resonant frequency of the resonant structure, one could also apply a voltage to the coil 200 while measuring the current. It should be noted that the positioning of magnetic material in a resonant structure near a coil or collection of coils alters the electrical properties of the coil(s). In particular, resonant frequencies can be measured. These changes in electrical properties of the coil(s) can be measured with signal processing devices which implement signal processing functions in analog circuits, digital circuits, and/or software controlled circuits. In particular, one or more of the resonant frequencies of the structure can be determined in this way. For example, the impedance of a single coil (such as 200 shown) will drop near a resonance of the structure incorporating a PM. An impedance analyzer or grid dip meter can serve to measure the changes in electrical properties of the coil. Also, the resonant structure/permanent magnet/coil system can be used to set the frequency of an electrical oscillator, as does a quartz crystal. Other signal processing devices are described below.

FIG. 2b depicts a mechanism for exciting motion along the directions 225. Other such mechanisms for exciting motion along 230 and 220 are shown in FIGS. 2c and 2d respectively.

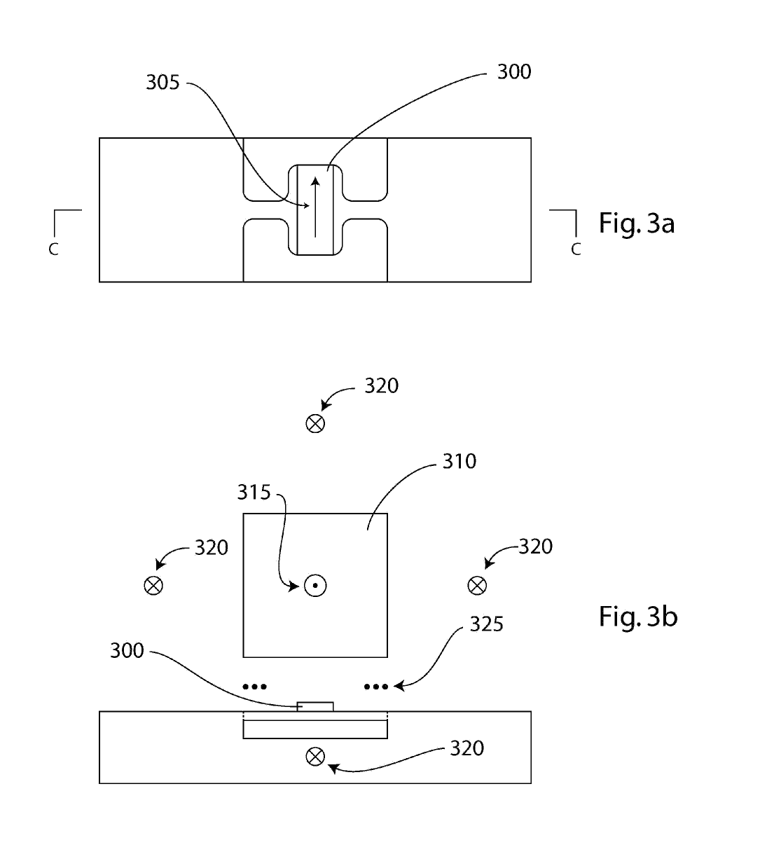

FIG. 3a depicts a system employing a soft magnetic material 300 wherein the magnetization arrow 305 is induced by an external magnetic field. FIG. 3b depicts a section of the same embodiment along cross section C-C. Further, FIG. 3b depicts a permanent magnet 310 magnetized at location 315 and producing a magnetic field into the page at locations 320 and others. In particular, the permanent magnet produces a magnetizing field for the soft magnetic material that magnetizes the material into the page in FIG. 3b and along the direction 305 in FIG. 3a. Once this soft material is magnetized, it can be excited by an AC current in a coil 325 in a fashion similar to those noted in FIGS. 2a through 2d.

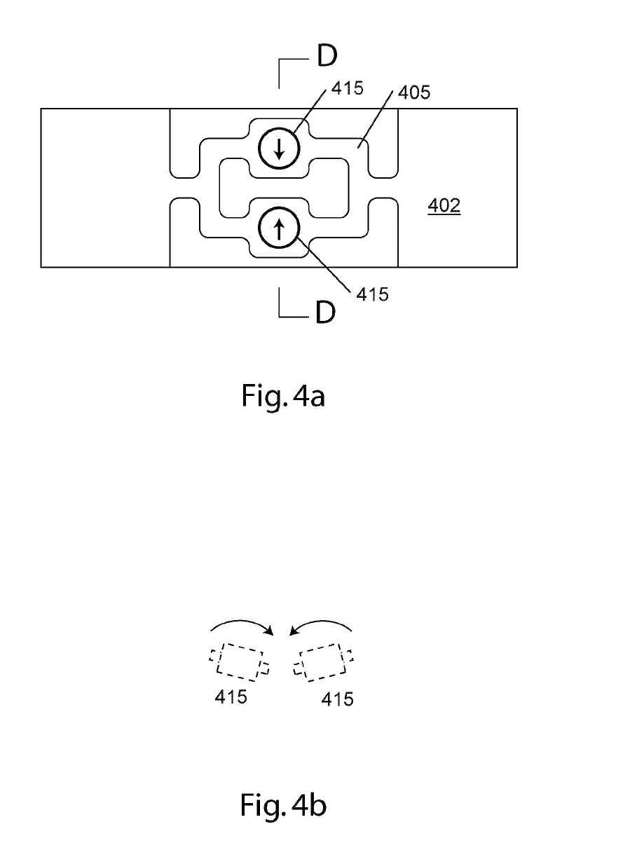

FIG. 4a depicts another embodiment of the invention wherein the mode shape of interest is symmetric, as shown in FIG. 4b which is taken across line D-D. The symmetry allows the vibration to occur with insignificant motion of the body 402. Thus, little energy is transferred to any structure supporting the body and the mode of interest will have a high Q because the losses to the surrounding structure are minimized. By analogy, a similar design principle is applied to musical tuning forks. A tuning force vibrates in a desired mode shape, but the handle of the fork does not, so tuning forks have a relatively high Q. The essential feature of these mode shapes is the insignificant motion of the supported body or supported points—this feature is referred to as dynamic balance. Geometric symmetry is common for a system with dynamic balance, but it is not essential. For example, the embodiment of FIG. 4a needs only one magnet and dynamic balance can be accomplished with an equivalent mass instead of the magnet. However, the embodiment of FIG. 4a employs opposing permanent magnet magnetizations including masses 455 and beams 405. The net dipole moment is nearly zero so that the system is not subjected to torque in an ambient magnetic field. This is beneficial if the sensor is to be used in magnetic medical imaging equipment (e.g., magnetic resonance imaging (MRI)) provided that the magnets are not demagnetized.

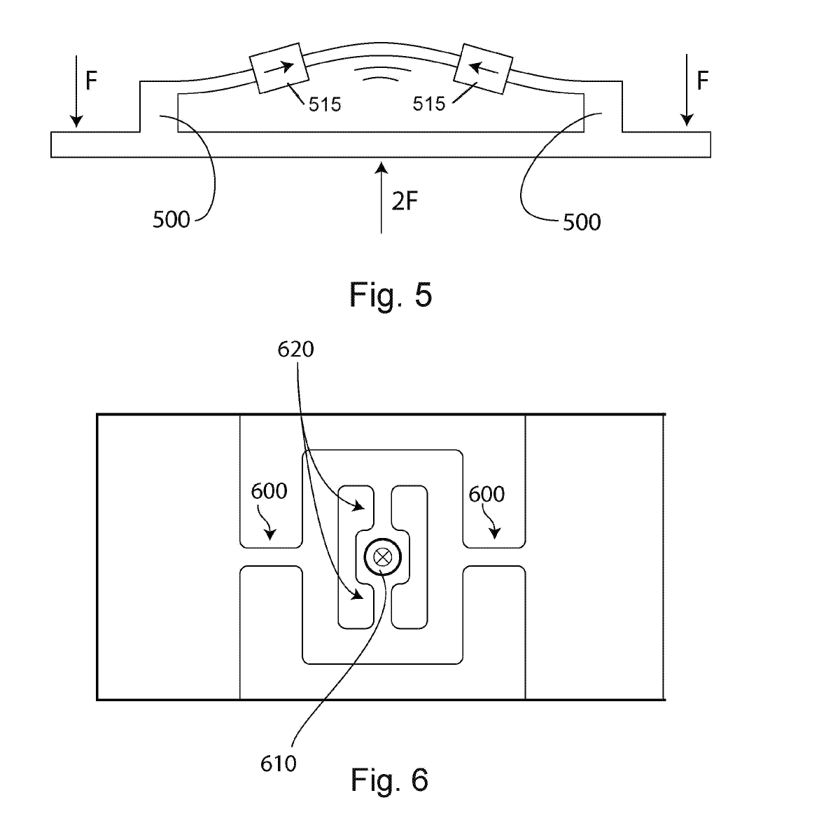

FIG. 5 is another embodiment shown in a snapshot during vibration. This design also has no net magnetic moment. It has multiple magnets 515 on a single beam and incorporates mechanical amplification of forces F and 2F. The mechanical amplification is accomplished in this elastic system through lever arms 500. In a force sensor, mechanical amplification converts (i.e., “focuses”) a higher fraction of the mechanical energy transmitted to the resonator by the external forces into mechanical strain energy in the resonant structure. This is done to maximize the frequency shift in the mode of interest. Here, the term mechanical amplification is used to mean this kind of focusing of mechanical energy.

FIG. 6 depicts an embodiment with an additional set of flexible beams 600 and 620, permanent magnet 610 and surrounding mass. The beams 620 are intended to undergo the largest vibrational motion. The beams 600 allow additional rotation of the permanent magnet so that the magnet can align with a large external magnetic field due to, for example, an MRI. In this way, torque transmitted to the body of the resonant structure can be reduced. In turn, when used in the human body, torque to supporting tissues is reduced.

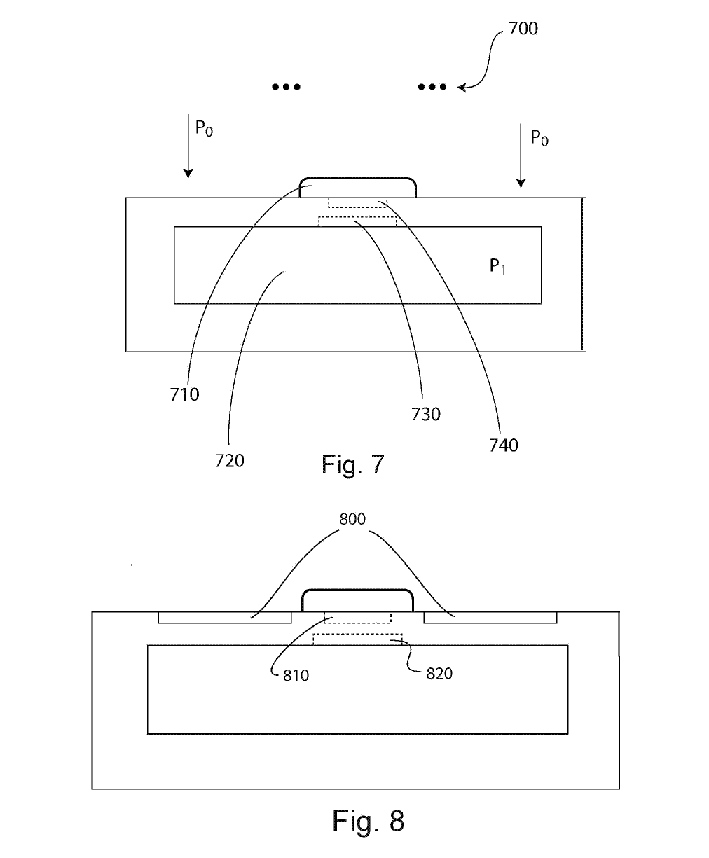

FIG. 7 depicts both a pressure sensor including a coil 700, sealed volumes 710 and 720 and two resonant structures 730 and 740 used in a differential mode. The embodiment includes sealed volumes to protect the resonant structures and create a reference pressure in volume 720. Resonator 740 is subjected to compressive loading when a pressure P0>Pi is applied and resonator 730 (operating in a different frequency range) is subjected to tensile loading. By knowing the temperature sensitivity of the frequencies of the resonant structures in this system, one can solve for the pressure difference P0−Pi independent of temperature. This is called a differential sensor. An exact or weighted difference of the frequency shifts might be used. In general, a weighted difference can be optimized to give the best rejection of temperature effects. Gas expansion effects when Pi is not zero (i.e., a vacuum) can also be accommodated in calculations. Further, more than two sensors can be used in differential mode. The frequency outputs of M resonant structures can be used to solve for M; different quantities provide that the M equations are not singular. Even if just one quantity is of interest, multiple sensors improve the estimate of that quantity.

FIG. 8 shows a modification of the pressure sensor of FIG. 7 to form a chemical sensor. Material 800 that preferentially adsorbs a chemical(s) of interest is incorporated into the sensor. If the chemical(s) are present, they are adsorbed and change the mechanical stress levels in the adsorbent material. This stress is transmitted to the resonant structures 810 and 820 and causes a shift in their resonant frequencies.

FIG. 9 shows the placement of a pressure sensor 900 incorporating the invention in the eye on an IOL haptic. Key features of the figure are the iris 910, an IOL 920, the lens capsule 930, the cornea 950 and a second IOL haptic 940. The pressure sensor can also be imbedded in the periphery of the IOL or attached to the tissues of the eye (not shown), including the iris 910. However, it is preferably placed outside of the optical path to the retina 960.

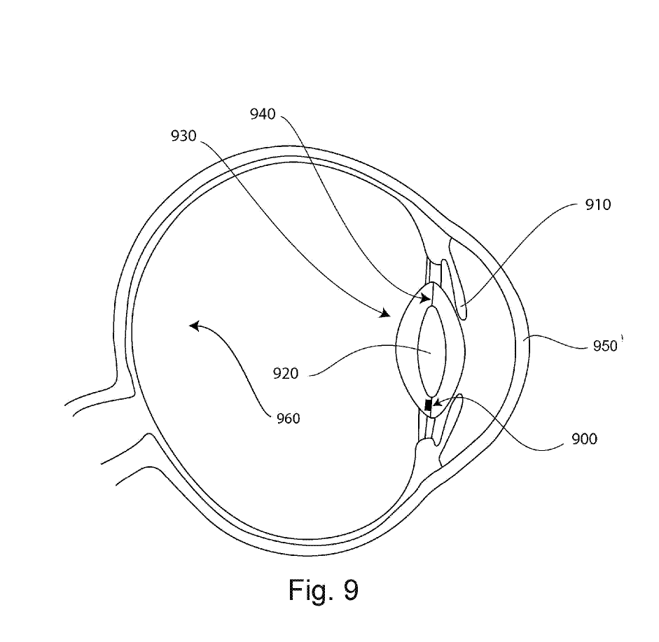

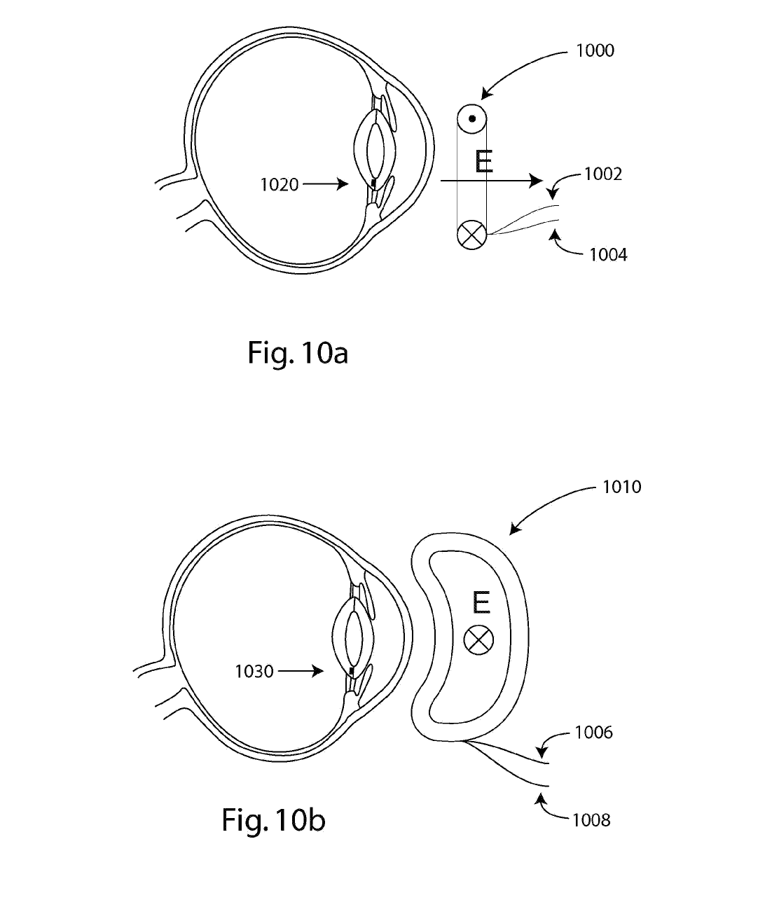

FIGS. 10a and 10b show possible placements of external coils 1000 and 1010 to interact with the magnetic material in the resonant structures of pressure sensors 1020 and 1030. FIG. 10a shows a geometry wherein a magnetic field is produced that is largely aligned with the optical path into the eye. The coil terminals are 1002 and 1004. FIG. 10b shows a geometry producing a field largely perpendicular to the optical path at the location of the sensor. The coil terminals are 1006 and 1008.

FIG. 11 depicts a signaling approach for communication with the pressure sensor. In particular, it depicts a sensor 1130 incorporating a resonant structure with an attached permanent magnet. The coil current is driven with pulsed tones. In between pulses, the coil 1100 is used to sense the oscillating magnetic field of the magnetic material. In this way, the high amplitude of the transmit signal does not interfere with the relatively weak signal produced by the vibrating magnet. The coil is alternately connected to the transmit circuitry and then to the receive circuitry with the analog transmit/receive switch as shown. The frequency of the pulsed tones is varied in order to search for a resonant frequency, or frequencies, of the sensor. This search is typically a coarse search to find the rough value of the frequencies and then fine searches to obtain accurate measurements of pressure. A useful feature of the signaling approach is the use of an analog switch to connect and disconnect the receive circuitry from the coil. Such an approach is referred to as a gated receiver.

FIG. 12 describes in some detail the structure of a possible transmit current. In order to detect a resonance at frequency fi, a total of Ni≧1 pulses (denoted at 1) of length Δi are transmitted with intervening quiet periods (denoted at 2) of the same length, Δi. Switching distortion due to finite switching speed can be minimized by choosing Δi to be an integer multiple of sine wave periods corresponding to the test frequency fi. The intervening quiet periods are used by a receiver subsystem to detect weak signals produced by the oscillating permanent magnet on the resonant structure. This signal takes the form of a periodically modulated sine wave and hence contains sidebands in the frequency domain in addition to a large component at the frequency fi. To avoid having the side bands excite resonances, Δi can be chosen sufficiently short so that the sideband is out of the frequency range of interest. Alternatively, the sideband effects can be interpreted by the receiver, or the transmit current can be modulated, to spread the energy in the sidebands. The advantageous features of this transmit signal is that it has a significant spectral component at fi and periods of zero output where the receiver can detect varying magnetic fields emanating from the resonant structure. Systems incorporating such signals are referred to herein as having pulsed drive signals.

FIG. 13 shows a signal processing system (SPS) incorporating a digital signal processor (DSP) 1310. The DSP “transmit software” produces a digital version of the pulsed signal (or equivalent) depicted in FIG. 12. This signal is converted to an analog signal with a digital-to-analog converter (D/A) 1315, filtered by a low-pass filter (LPF) 1320 to remove effects of time sampling and then processed by an amplifier (amp) 1325. The resulting current signal is transmitted to a coil 1300 when the analog switch 1330 in the “up” position. In between pulses, the switch is in the “down” position. Magnetic signals from the resonant structure are communicated with the DSP via an amp 1345, an anti-aliasing filter 1350, and an analog-to-digital converter (A/D) 1355. Alternative approaches to signal processing involve continuous coil impedance measurements using a grid dip meter or equivalent. There are numerous ways of implementing the signal processing system so long as there is an excitation of the resonant structure and it interprets the vibrational motion of the resonant structure to estimate at least one resonant frequency and/or a sensed quantity.

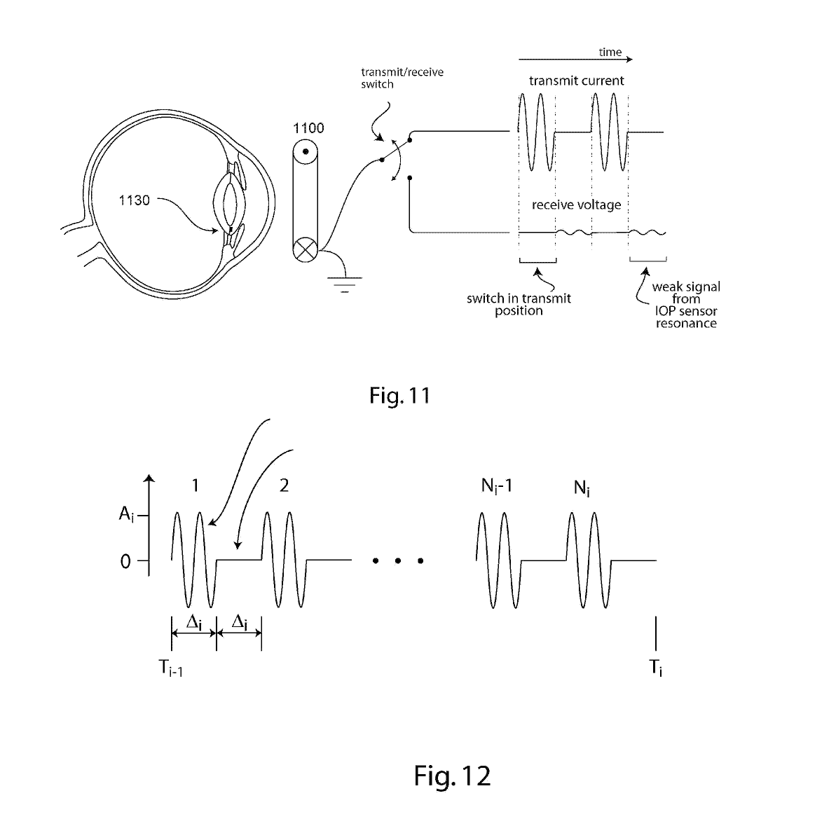

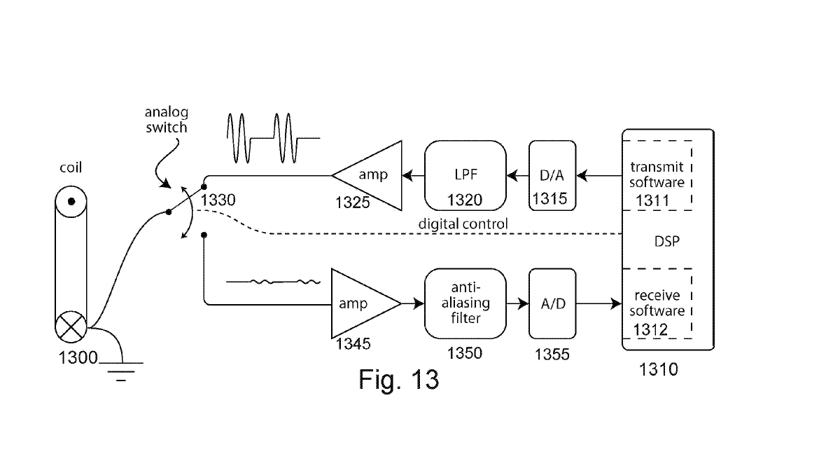

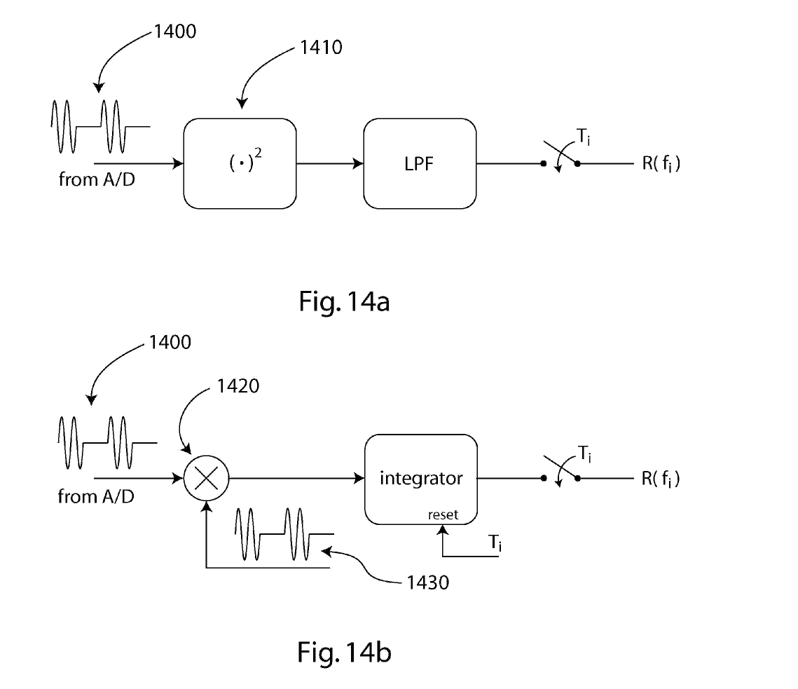

FIGS. 14a and 14b depict two block diagrams for the receiver software represented inside the DSP in FIG. 13. In general terms, the software is searching for the frequency(s) where the receiver gets a large response from the coil(s) near the sensor. The receive signal is represented by 1400 in FIGS. 14a and 14b. A simple processing technique is depicted in FIG. 14a and involves rectification (conversion to DC) using a squaring function 1410 followed by a low-pass filter (LPF). The LPF output is sampled at the end of the fi pulse train to create the response at this frequency denoted R(fi). Because this response depends on the signal amplitude and length of the pulse train, some normalization may be required. The rectification is shown with a squaring circuit, but other functions work as well, including an absolute value function and a time-synchronized demodulator which switches at the zero crossings. FIG. 14b shows the so-called matched filter approach to signal processing. The amplified receive signal is multiplied 1420 with the expected receive signal 1430 and integrated. At the end of the pulse train, at time Ti, the integrated response is sampled to form R(fi) and the integrator is reset.

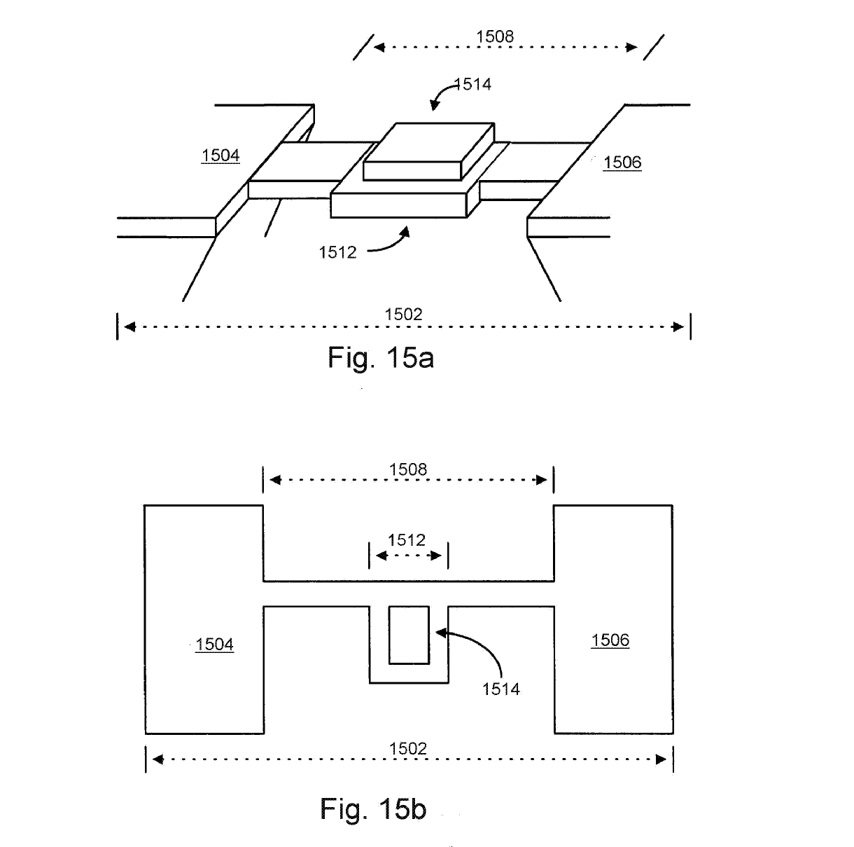

FIG. 15a illustrates an alternative preferred embodiment of resonant structure 1502 that is used in the construction of a magnetically driven resonator. As illustrated by FIG. 15a, the resonant structure includes a proximate portion 1504 and a distal portion 1506. As mentioned above, the resonator is a device that contains an element that vibrates at its mechanical resonant frequency and, as such belongs to the class of oscillators for which energy alternates from one form of storage to another, for example from kinetic to potential energy.

The resonant structure 1502 is formed such that a resonant bridge 1508 extends between the proximate 1504 and distal 1506 portions of the resonant structure 1502. It should be noted that, although a bridge structure is shown in FIG. 15a, those skilled in the art will recognize that a variety of mechanically resonant structures, including strings, cantilever beams, etc., may be utilized. A central bridge portion 1512 is located central to the resonant bridge 1508 and extends horizontally from one side of the resonant bridge 1508, perpendicular to the central axis of the resonant bridge 1508 and on the same plane as the proximate 1504 and distal portions 1506 of the resonant structure 1502. FIG. 15b is a top view of the resonant structure 1502 that better illustrates the resonant bridge 1508 in accordance with the present invention.

One skilled in the art will appreciate that the central bridge portion 1512 need not be located exactly central to the resonant bridge 1508 but may instead be located closer to the proximate 1504 or distal 1506 portions of the resonant structure 1502. Basically, positioning of the central bridge portion 1512 must allow for accurate measurement of changes in resonant frequency of the resonant bridge 1508 when the resonant structure 1502 is subject to mechanical stress. Therefore, the central bridge portion 1512 may be located anywhere on the resonant bridge 1508, as long as accurate measurement of changes in resonant frequency is possible.

A solid hard magnet material (magnet) 1514 is located on a top surface of the central bridge portion 1512 of the resonant bridge 1508 such that the solid magnet 1514 in turn, can be used to drive excitation of central bridge portion 1512 of the resonant bridge 1508, and therefore, the entire resonant bridge 1508. In accordance with the preferred embodiment of the invention bonded ferrite, or other hard magnetic material, in a polymer matrix has been selected as the solid magnet material in order to avoid high temperature fabrication steps and to avoid difficulties that may be associated with bonding a solid magnet to a resonator. Such difficulties may include alignment and bonding of a conventional magnet on a relatively delicate flexure. However, the assembly and bonding of a conventional magnet to the structure does have the advantage of being able to use a magnet with excellent magnetic properties and could be used in an alternate embodiment of the invention. As known in the art, a bulk magnet may also be used as the solid magnet. One skilled in the art will appreciate that the solid magnet 1514 may be fixed to the resonant bridge 1508 by many different means, such as, but not limited to, bonding the solid magnet 1514 to the central bridge portion 1512 of the resonant bridge 1508 using a means such as an adhesive; attaching to the central bridge portion 1512 of the resonant bridge 1508 by means such as a clamp; or connecting to the central bridge portion 1512 of the resonant bridge 1508 by means of screen printing, or by means of using magnetic fields (for example, emanating from a clamping magnet on the underside of the resonant bridge 1508).

In accordance with one embodiment of the present invention, the solid magnet 1514 is subjected to a magnetic field such that the magnetization vector of the solid magnet 1514 is permanently fixed in a single direction. Thereafter, the solid magnet 1514 is attached to the central bridge portion 1512 of the resonant bridge 1508 such that the direction of the magnetic field of the solid magnet 1514 is parallel to the central axis of the resonant bridge 1508, either from the proximate portion 1504 to the distal portion 1506 of a resonant structure 1502, or vice-versa. The resonant structure 1502 can be constructed of a single crystal material such as, but not limited to, single crystalline silicon or quartz. As one skilled in the art will appreciate, the resonant structure 1502 need not be limited to being constructed by a single crystal material, but instead may be constructed of any material that is capable of resonating at a high amplitude without excessive consumption of power. Because both materials are anisotropic, anisotropic etchants can be used to obtain desired shapes. A main advantage to processing silicon is the several different fabrication techniques, well-known in the micro-machining art, for the precise control of the geometry of the structure. Although polycrystalline silicon does not show mechanical properties quite as high quality as many single crystal materials, it has characteristics which can be used to make the resonator structure 1502 with very precisely controlled dimensions due to the standard process of deposition and stress control of fine grained polycrystalline silicon layers.

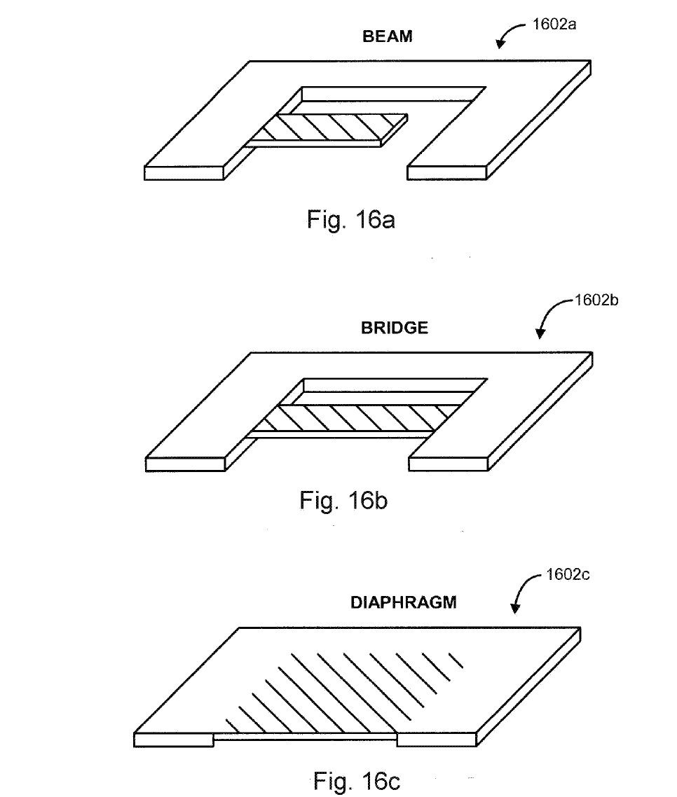

FIGS. 16A, 16B, and 16C illustrate three common shapes that exist for building resonators including the beam shape 1602a, the bridge shape 1602b, and the diaphragm shape 1602c. Each of these shapes, or structures, has several different resonant modes, where each mode has its own displacement pattern, resonant frequency, and quality factor. As known in the art, a quality factor is the ratio between the total energy stored in the system and the energy losses in the vibrating element. It can also be calculated from the curve of amplitude of the vibration element versus its frequency by taking the resonant frequency, divided by the frequency bandwidth, at the 3 dB amplitude points. In accordance with the illustrative embodiment of the invention, as mentioned hereinabove, the bridge shape is used in constructing the resonator structure.

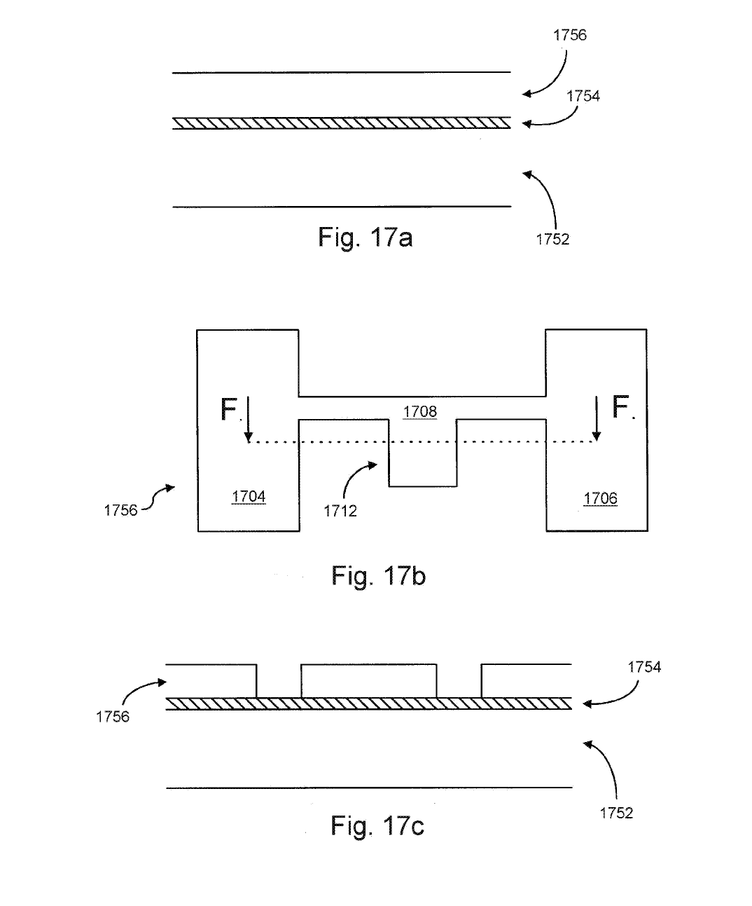

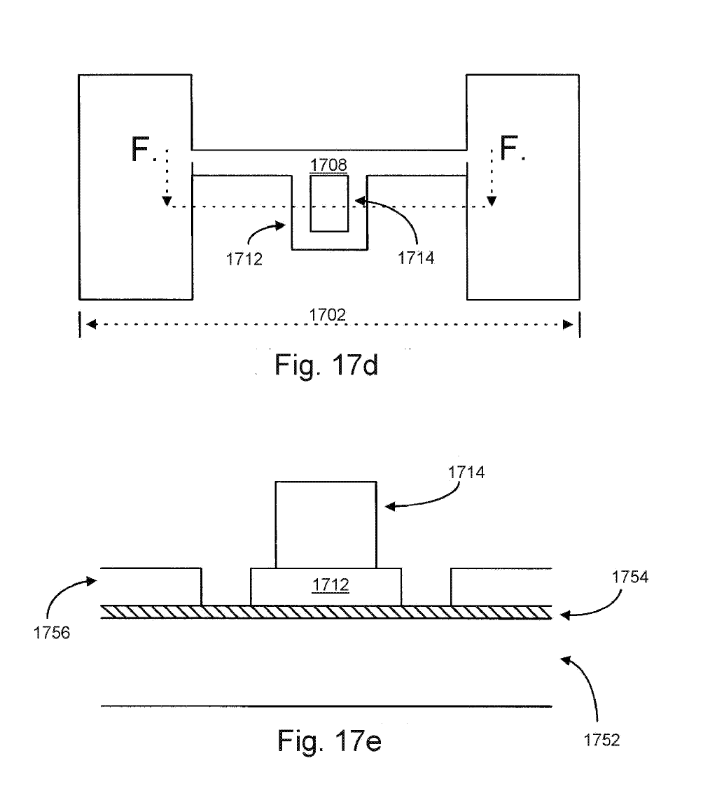

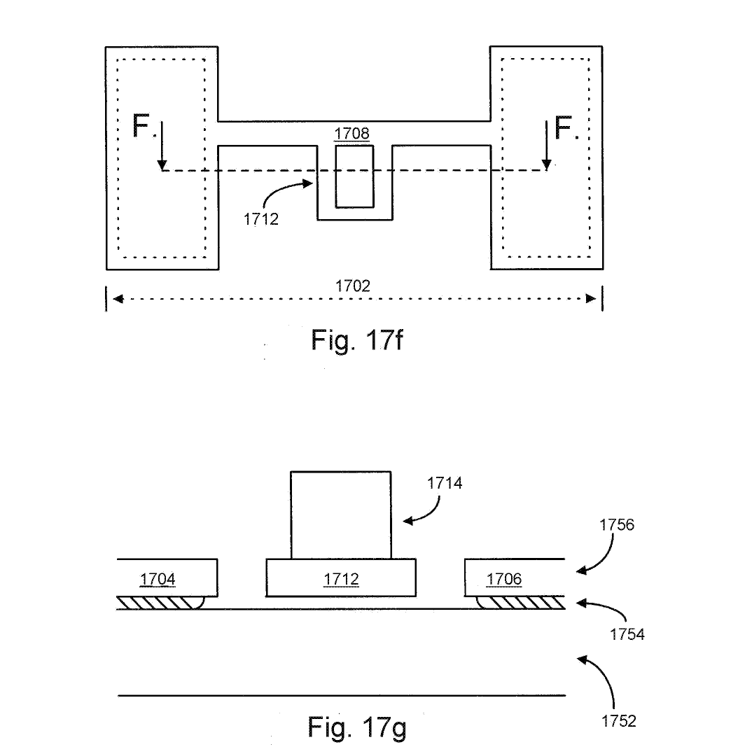

Fabrication of the magnetically-driven resonator is described with reference to FIGS. 17a through 17f described hereinbelow. As illustrated by FIG. 17a, and in accordance with an embodiment of the invention, the magnetically-driven resonator is constructed from silicon located on insulator wafers that include a lower layer 1752, a central layer 1754, and a top layer 1756. Preferably, the lower layer 1752 silicon, the central layer 1754 is silicon dioxide, and the top layer 1756 is silicon. A single crystal silicon has been selected as the resonator material due to its excellent mechanical properties and for its micro-machined simplicity compared to elements such as quartz. It should be noted, however, that alternate materials may be used as known by those skilled in the art, and, as such, the use of silicon described herein is merely an example is usable material.

The silicon is then patterned as illustrated by FIG. 17b, which shows a top level view of the top layer 1756 of the resonant structure where the top layer 1756 of the silicon includes the proximate portion 1704, the distal portion 1706, the resonant bridge 1708, and the central bridge portion 1712. FIG. 17c provides a cross section view of the resonant structure illustrated by FIG. 17d, along the axis F-F. As described hereinabove, with reference to FIG. 17d, the central bridge portion 1712 of the resonant bridge 1708 is located central to the resonant bridge 1708 and extends horizontally from one side of the resonant bridge 1708, perpendicular to the central axis of the resonant bridge 1708, and on the same plane as the proximate 1704 and distal portions 1706 of the resonant structure 1702. As known to one skilled in the art, multiple patterning methods may be used in order to pattern the silicon in accordance with the preferred embodiment of the invention including, but not limited to, dry and wet etching.

After patterning the silicon in order to shape the resonant structure, the solid magnet 1714 is preferably screen-printed on the central bridge portion 1712 of the resonant bridge 1708. It will be appreciated that the solid magnet 1714 may be fixed to the central bridge portion 1712 of the resonant bridge 1708 by using any other method known in the art that will allow the solid magnet 1714 to remain on the central bridge portion 1712 of the resonant bridge 1708 during vibration of the resonant structure. FIGS. 17d and 17e illustrate the bond between the solid magnet 1714 and the central bridge portion 1712 of the resonant bridge 1708 wherein FIG. 17d is a top view illustration of the bond. As illustrated, FIG. 17e is a cross section of FIG. 17d along the axis F-F.

In accordance with the preferred embodiment of the invention, the patterned top layer 1756 of silicon corresponding to the resonant bridge 1708 and the central bridge portion 1712 of the resonant bridge 1708 is then released from the lower layer 1752 of silicon by removing the central layer 1754 of silicon dioxide. FIGS. 17f and 17g illustrate removal of the central layer 1754, wherein FIG. 17f is a top level view of the patterned top level having the beginning of the silicon central layer 1754 represented by dotted squares. Further, FIG. 17f is a cross-sectional view of FIG. 17e taken across line F-F. Preferably, wet or dry isotropic etching of the sacrificial silicon dioxide is performed to free the resonant bridge 1708 and the central bridge portion 1712 of the resonant bridge 1708 from the central layer 1754 of silicon dioxide. As illustrated by FIGS. 17f and 17g, the proximate 1704 and distal portions 1706 of the resonant structure 1752 remain connected to the lower layer 1752 of silicon via the central layer 1754 of silicon dioxide, such that the proximate 1704 and distal 1706 portions of the resonant structure support the resonant bridge 1708 and the central bridge portion 1712 of the resonant bridge 1708. This process allows the resonant bridge 1708 and the central bridge portion 1712 of the resonant bridge 108 to vibrate while being supported by the proximate 1704 and distal 1706 portions of the resonant structure.

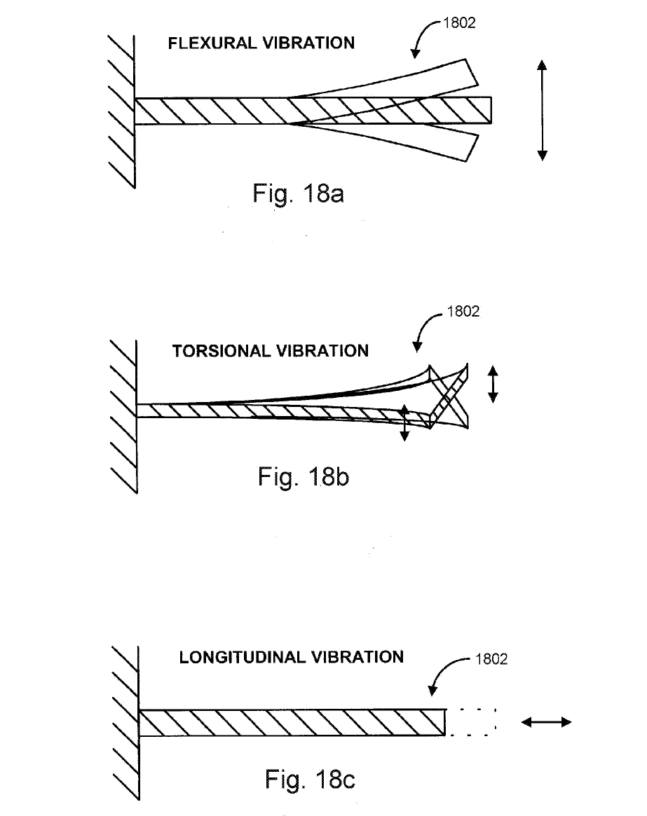

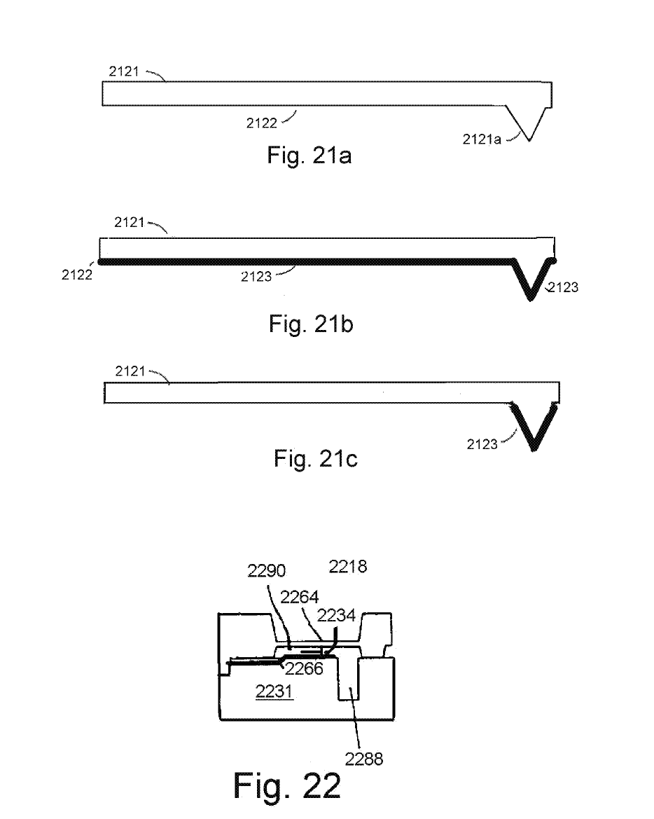

When vibrating, the resonant structure, including the bridge 1708 and central bridge portion 1712 of the resonant bridge 1708, may vibrate in numerous different modes. As shown by FIGS. 18A, 18b, and 21C, a resonant structure may vibrate in a flexural vibration mode, a torsional vibration mode, or a longitudinal vibration mode. Those of ordinary skill in the art will appreciate that a resonant structure 1802 may also vibrate in other modes known in the art, and, as such, the aforementioned vibration modes are merely provided as examples. Preferably, the resonant structure 1802 vibrates in torsional mode.

Therefore, a number of alternative embodiments are possible. Optionally the device is made of cantilever-type beam(s) with one end free to vibrate. However, a similar device may be constructed using beams of other configurations, such as simply supported beam(s) wherein both ends are supported, free to rotate; or beam(s) with both ends fixed, not free to rotate; with one end fixed and one end supported and free to rotate; and other simple and compound beam structures and combinations, such as triangular beam(s) having two corners fixed and the third corner free.

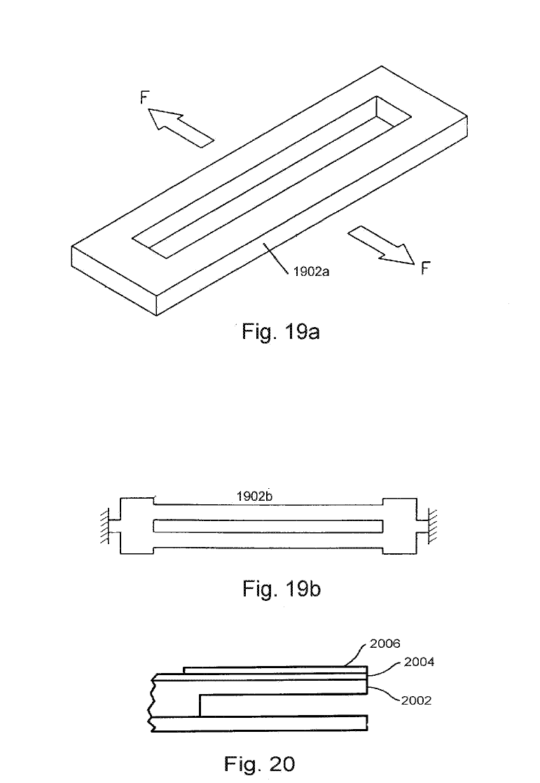

The mechanical resonant structure can be relatively complex, since it is essentially aimed at enhancing as much as possible, for an equal variation in the applied pressure P, the corresponding variation in the resonance frequency. For example, one structure, which is typically used in the state of the art, is the so-called DETF (Double Ended Tuning Fork) structure, shown schematically in FIG. 19a. According to this structure, the resonant structure 1902a includes two oscillating beams. In order to optimize mechanical performance, the beams may have a very small thickness and width (a few microns) and a relatively significant length (hundreds of microns).

The resonant structure according to a preferred embodiment of the present invention, is formed by a balanced resonator which is capable of minimizing the constraint reactions caused by the oscillations of the resonator, thus reducing the effect of the damping actions at the coupling points between the resonator and the diaphragm. In the balanced resonator, the beams vibrate in phase opposition and at the constrained ends the reactions to the motion of the two beams partially compensate each other, with a consequent lower dissipation of energy with respect to the case of a single vibrating beam. The balanced structure also allows several additional advantages, such as greater stability with respect to external influences, higher resolution, and reduction of the effect of long-term drifts.

Advantageously, as shown in the embodiment in FIG. 19b, the DETF resonator 1902b is configured so as to have at each end two lateral protrusions and a connecting portion which are respectively wider and narrower than the central portion of the resonant structure. It is also envisioned a resonant structure of three or more parallel beams.

The resonance frequencies of a beam occur at discrete values based on the geometrical and mechanical properties of the beam and the environment in which it is located. The efficiency of resonance is measured by the quality factor (or Q-factor), where large Q-factors correspond to high efficiency. Cantilever beams have and especially high Q-factor. Moreover, microcantilevers, which are only a few hundred microns in length, are also very straightforward to produce using MEMS fabrication technologies. Thus, it is desirable to make a high-Q cantilever that exhibits a broad range of resonance frequency under a narrow range of mechanical stress. There are several approaches by which the resonance properties of a cantilever can be varied. The approach involves the application of a stress sensitive film to the micro-beam surface. Young's Modulus for many polymers varies with applied stress due to changes in bond length of the constituent molecules.

If the cantilever is coated with or comprises a stress-sensitive material, the stiffness will be changed as the beam to a larger degree than without a stress-sensitive material. The stress-sensitive material may preferably be selected from but not limited to the group consisting of metals, metal alloys, dielectric materials, polymeric materials and combinations thereof. Specific examples of such polymeric materials include but are not limited to such polymers as polycarbonate of visphenol, poly[N,N′-(p,p′-oxydiphenylene) pyromellitimide], poly(vinyl chloride), and the like. Many other polymers are known that perform as described herein. A method for varying cantilever resonance frequency is shown in FIG. 20 which represents a side view of a magnetically-coupled cantilever. In FIG. 20, a cantilever 2002 has a ferromagnetic coating 2004 and a stress-sensitive coating 2006 applied to one surface. The cantilever 2002 may consist of any of a number of dielectric materials, such as silicon nitride or silicon dioxide, while the ferromagnetic element 2004 may preferably be composed of metals such as iron or nickel or some other ferromagnetic material.

Adequate magnetic films can be deposited on microbeams of a few hundred Angstroms of rare-earth magnetic alloys (magnetic materials), such as Neodymium-Iron-Boron (Nd/Fe/Bo). Other magnetic alloys with suitable moments are samarium cobalt and Alnico, an alloy of aluminum, nickel, and cobalt. They may be used in combination, if desired. Such materials are readily capable of magnetization in the presence of a magnetic field of sufficient magnitude.

In accordance with an alternative preferred embodiment of the present invention, magnetic material is formed into a sputter target for use in a sputter deposition system similar to those used in the semiconductor industry for the deposition of metallic films onto silicon wafers, and more specifically according those methods disclosed in U.S. Pat. No. 5,866,805 (Han et al.). Accordingly, the entirety of the methods disclosed in U.S. Pat. No. 5,866,805, to the extent applicable, is incorporated to the present invention herein.

Referring now to FIGS. 21a through 21c, there can be seen an alternative embodiment of a cantilever 2121 and tip 2121a that has been coated along cantilever 2121 with photoresist layer 2122. According to this embodiment, a photoresist layer 2122 does not extend over tip 2121a. After the application of photoresist layer 2122, ferromagnetic layer 2123 is applied to the entire cantilever 2121 and tip 2121a as shown in FIG. 21b. Subsequently, cantilever 2121 and tip 2121a are treated to remove ferromagnetic layer 2123 from cantilever 2121, but not from tip 2121a as there was no photoresist on tip 2121a, as shown in FIG. 21c. This embodiment avoids residual magnetic material over the length of cantilever 2121 in accordance with similar methods disclosed in U.S. Pat. No. 6,676,813 (Pelekhov et al.) which, to the extent applicable, is incorporated into the present invention.

According to a preferred embodiment of the present invention, the diaphragm is bonded to the substrate preferably via a hermetic sealing process. Alternatively, a post-bond coating of the entire sensor may be used to establish a hermetic interior. In either situation, steps are taken to minimize the residual gas pressure within the sensor after a hermetic seal is established. Once the initial hermetic seal is achieved, gas may be trapped in the interior of the sensor due to continued outgassing of the interior surfaces and/or the bonded regions. The pressure of the residual gas will increase within the interior chamber of the pressure sensor as the diaphragm deflects during normal operation. This residual gas may affect the overall sensitivity of the pressure sensor. Additionally, the residual gas will expand and/or contract with changes in the temperature of the sensor itself, causing signal drift.

To compensate for the various negative effects of any residual gas, the pressure sensor 2218 of the present invention is provided with a displacement cavity 2288. This displacement cavity 2288 is generally seen in FIG. 22 and is in communication either directly or through a small connecting channel with the interior chamber 2290 of the pressure sensor 2218, defined between the diaphragm 2264 and surface 2266. The displacement cavity 2288 is sized such that the total internal sensor volume, the combined volume of the displacement cavity 2288 and the interior chamber 2290, varies minimally with deflection of the diaphragm 2264 over its operational range of displacement. By minimizing the overall change in volume with deflection of the diaphragm 2264, the effect of the residual gasses are minimized and substantially eliminated. In such embodiment of the present invention, the volume of the displacement cavity 2288 is approximately ten times greater than the volume of the chamber 2290. To further reduce temperature induced drift and to increase the sensitivity of the device, lower pressures within the internal volume 2290 should be used.

Referring further to FIG. 22, the substrate 2231 may be part of a silicon diaphragm in a pressure sensor, and thus the pressure causing deflection of the diaphragm. The substrate 2231 may also be utilized as a strain transducer by gluing or otherwise tightly affixing it to a larger structure which is undergoing strain. The strain of the underlying structure is transmitted to the substrate 2231 and thence to the resonating beam 2234 to thereby affect the resonant frequency of the beam. The transducer structure may be made quite small, and is formed in a way which is compatible with microelectronic circuit processing techniques. For example, the beam 2234 may have a length in the range of a few hundred microns, e.g., 200 microns, with the width being in the range of a few tens of microns and thickness of the beam 2234 in the range of a few microns, e.g., 1-2 microns.

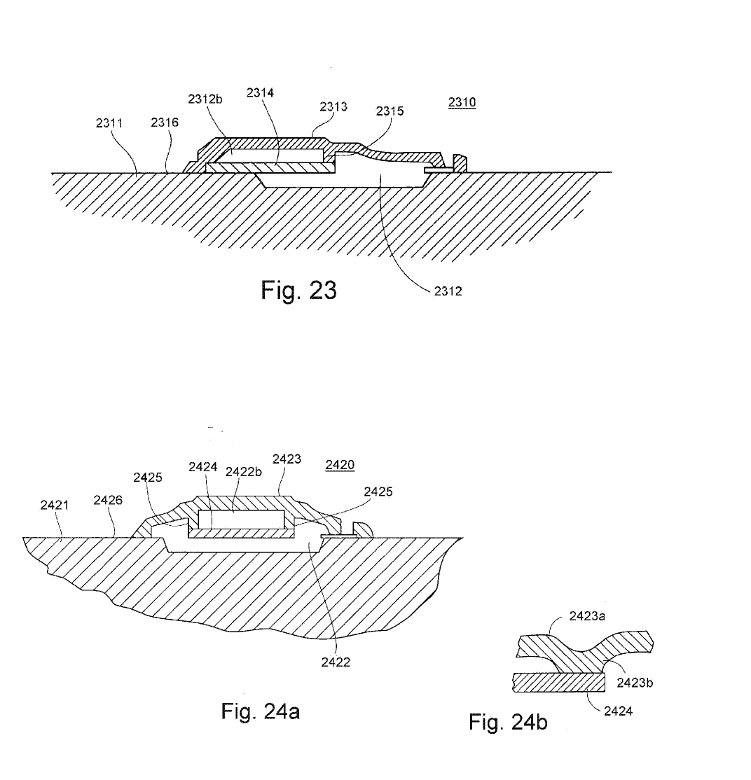

Referring to FIG. 23, shown is a preferred embodiment whereas at least one resonant microbeam is suspended by the fixable diaphragm. FIG. 23 shows a cross section of an embodiment of the present thin film resonant microbeam sensor device 2310 according to the present invention. Device 2310 includes a substrate 2311 of silicon, in which there has been formed a depression by surface micromachining, sacrificial oxide, etching and reactive sealing. Covering the depression there is a diaphragm 2313 of amorphous silicon. In this embodiment, the diaphragm structure is slightly elevated from the upper surface 2316, and thus a vacuum cavity 2312, 2312b is formed between diaphragm 2313 and substrate 2311. It would of course be conceivable to make a structure where the membrane is located essentially in the same plane as the surrounding substrate.

Within the cavity 2312 a resonant beam member 2314 is provided suspended at one end of its ends by a suspension member 2315 connecting the beam with the diaphragm 2313, and at its other end attached to the substrate 2311. Thus, the entire surface of the beam 2314 is spaced from both the diaphragm 2313 and the substrate, respectively by a certain selectable distance, by providing suspensions 2315 of appropriate length, which is an advantageous aspect of the invention, because it enables the sensitivity of the sensor to be controlled and increased. For instance, both the distance above the beam 2314 and below is selectable, the distance below by controlling the depth of the cavity. Thus, the beam 2314 is free to vibrate inside the cavity 2312. It should be noted that the area indicated with reference numeral 2312b is part of the cavity 2312 and is in complete communication therewith. Pressure applied to the top side of the diaphragm 2313 deforms the diaphragm and causes the beam 2314 to stretch; thereby changing its resonance behavior, e.g., the resonance frequency of the beam will change.

The beam can have a number of different shapes. It could be rectangular, triangular hexagonal, octagonal, circular, etc., just mention a few possibilities, and it may also comprise slots of various shapes. It should also be noted that the edges of the beam member 2314 is spaced from the walls in the cavity 2312 and thus the edges of the beam are free to move except at the suspension points.

FIG. 24a shows another embodiment of the sensor device. It includes the same basic elements as the embodiment in FIG. 23—i.e., a substrate 2421, a depression forming a cavity 2422, 2422b, a diaphragm structure 2423, and a resonant beam member 2424. However, in contrast to the embodiment of FIG. 23, the resonant beam member 2424 is suspended at both its ends by suspension elements 2425 connecting with the diaphragm 2423. In all other respects, the structure of this embodiment is the same as that of FIG. 23. The fact that the beam 2424 is entirely suspended by the diaphragm has certain advantages.

It should be noted that the suspension elements 2415, 2425 although they are referred to as elements, may form a part of the diaphragm. Either as indicated in FIGS. 23 and 24, where they form separate projections depending from the diaphragm, or by shaping the diaphragm so as to form an attachment connecting the microbeam to the diaphragm in a spaced apart relationship. This is illustrated in FIG. 24b, wherein a diaphragm 2423 is formed with a bulge like portion 2423b attaching to a beam member 2424.

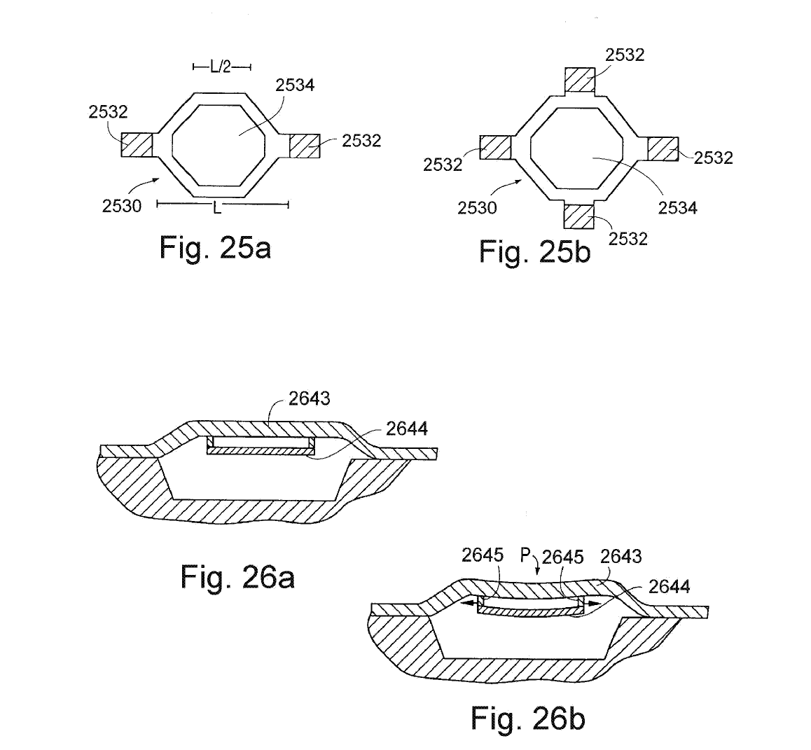

In FIGS. 25a and 25b various possible designs of the beam member are shown. FIG. 25a illustrates an embodiment of a beam 2530 and magnetized structure 2534 having two suspension points 2532, one of which may be attached to the substrate (as in FIG. 24), the other to the diaphragm via a suspension element (such as element 2425 in FIG. 24a). Alternatively both suspension points may be attached to the diaphragm. The specific shape of the diaphragm is not critical, although the geometry indicated in FIG. 25a has certain advantages. If the beam according to this embodiment is made longer but maintaining the width thereof, it will have a lower resonance frequency, thus providing for better separation of diaphragm and beam frequencies, but instead the sensitivity will be reduced. Thus, there will always be a trade off between desired frequency and the desired sensitivity.

FIG. 25b illustrates an embodiment having four points of attachment 2532 and magnetized structure 2534. In principle all possible combinations of attachments are possible, e.g., all four points attached to the substrate, one or more attached to the substrate and the rest suspended by the diaphragm, or all four points attached to the diaphragm. In this embodiment, the resonance frequency will increase as much as three times. An advantage of this embodiment is that one can obtain different vibrations in different directions. This may be used to advantage by enabling pressure detection and temperature detection to be performed at the same time. Although this embodiment will have somewhat lower pressure sensitivity compared to the embodiment of FIG. 25a, there are some advantages with it. Thus, the beam will become symmetric within the sensor, whereby the diaphragm will have a better appearance; the beam will be slightly more isolated from the environment; the sensitivity to the method of manufacture is less; the beam is smaller, which could mean easier excitation, since there is a smaller mass.

As can be seen in FIGS. 23, 24a, and 24b, the suspension elements constitute the coupling between diaphragm and beam. Thus, a deflection of the diaphragm when exposed to pressure will cause the suspension elements to be urged towards the periphery. In FIGS. 26a and 26b this deflection is shown schematically. FIG. 26a shows a diaphragm 2643 unaffected by pressure, and FIG. 26b shows a pressure P being exerted on the diaphragm 2643. When the diaphragm 2643 bends down, the suspensions 2645 must follow the movement of the diaphragm and thereby they exert a pulling force on the beam 2644 in opposite directions, whereby the beam 2644 will be subject to a stress and tend to become elongated, which will cause its resonance frequency to shift. The stress induced in the beam 2644 by a given pressure will of course increase if the leverage provided by the suspension elements is increased. The relevant parameter for the lever action is the “average” distance between the center line of the diaphragm and the beam.

The leverage is optimized by controlling the length of the suspensions simply by making the suspensions longer. However, there is an optimum for the sensitivity as a function of suspension length, for a given set of other parameters. The provision of leverage by the suspension of the beam is a very important aspect of the invention, and provides significant advantages.

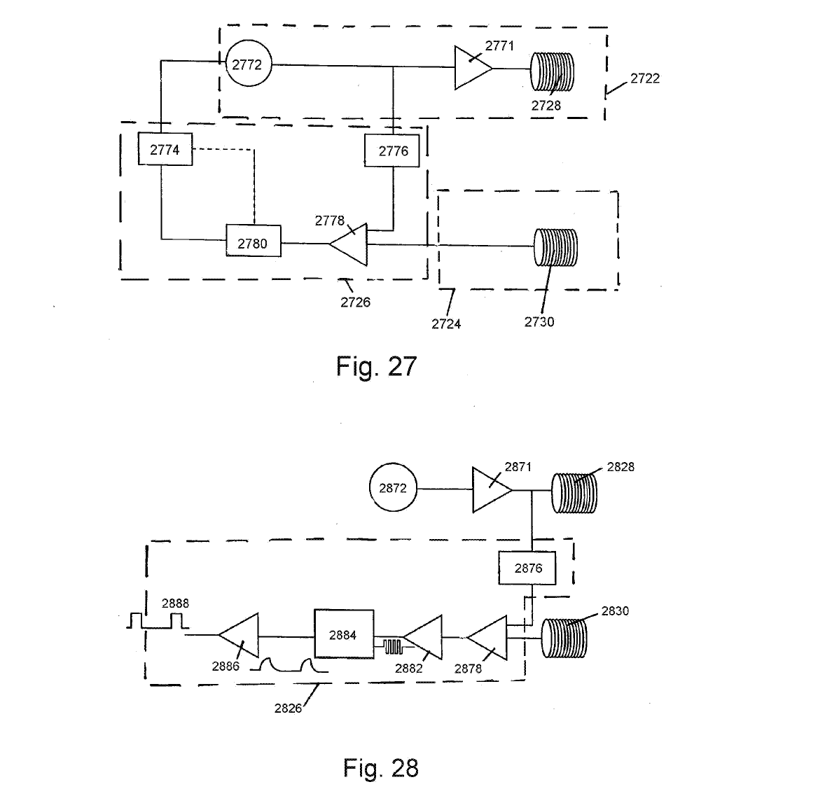

FIGS. 27 and 28 are alternative embodiments of the SPS shown in FIG. 13. Referring to FIG. 27, there is shown a block diagram for a first alternative data interpretation system including an excitation block 2722, a receive block 2724, and an interpretation block 2726. The excitation block includes an excitation coil 2728, and the receive block includes a receiving coil 2730. The interpretation block includes receiving circuitry for the continuous data interpretation—i.e., monitoring of pressure. Alternatively, the excitation coil 2728 and the receiving coil 2730 may be reduced to functions of one coil. In this alternative embodiment, the coil may alternate in a time division multiplexed manner between an excitation function and a receiving function. The interpretation block 2726 includes a controller 2774. The controller 2774 is preferably a microprocessor or a digital signal processor that controls the excitation oscillator 2772 that is connected to an excitation amplifier 2771, to detect peak responses, and to convert the peak responses from resonant frequency to the sensed pressure. The controller 2774 preferably sets the frequency that the excitation oscillator 2772 outputs.

Signal from the excitation oscillator 2772 is current amplified and output to the excitation coil 2728. The output is exposed to the magnetically-driven resonator (as previously discussed). The pickup coil 2730, which preferably is in a coaxial manner with the excitation coil 2728, receives a first signal directly from the excitation coil, and a second signal from the magnetically-driven resonator 2720.

The data interpretation block 2726 has a cancellation circuit 2776. The cancellation circuit 2776 has a canceling coil therein (not shown). The canceling coil (not shown) preferably is wrapped in an opposite direction relative to pickup coil 2730, or alternatively is a phase shifted differencing amplifier. The resultant output from a pickup amplifier 2778 (that is connected to the pickup coil 2730 and the cancellation circuitry 2776) is substantially solely from the magnetically-driven resonator 2720.

The data interpretation block 2726 has a detector 2780. The detector 2780 may be any circuitry known in the art that allows the controller 2774 to measure peak amplitude of the output of the pickup amplifier 2778. The detector 2780 may alternatively be a filtered rectifier, a peak detecting sample, a hold circuit, an analog to digital converter run by the controller 2774 or any other type of amplitude demodulating circuitry. In another embodiment, the controller 2774 may control the detector 2780 in more digitally controlled embodiments.

Referring to FIG. 28, there is shown another or second embodiment of the data interpretation system for a discrete type resonant sensor of the present invention. The oscillator 2872 implements a single excitation frequency. The oscillator's output is a current that is amplified by the excitation amplifier 2871 to drive the excitation coil 2828 and emit the electromagnetic field in the sensor. In this embodiment, the pickup coil 2830 is formed as a sensor receiver coil that picks up the magnetic field due to both the excitation coil 2828 and the magnetically-driven resonator. The data interpretation block 2826 includes a cancellation circuit 2876 that is connected between the pickup amplifier 2878 and the excitation coil 2828. The cancellation circuit 2876 removes any artifact of the excitation coil 2828. The cancellation circuit 2876, as in the embodiment of FIG. 28, may be a canceling coil (not shown) wrapped in the opposite direction from that of the pickup coil 2830, a differencing amplifier, or alternatively any other suitable device known in the art.

An alternating current output of the pickup amplifier 2878 is run through a band pass filter 2882 and may be centered at an expected ideal resonant frequency. This alternating current output outputs a band pass filtered signal. The band pass filtered signal is made unipolar by a rectifier collectively shown with the low pass filter as reference numeral 2884. The rectifier 2884 may be a full or a half wave rectifier. The data interpretation system 2826 has a low pass filter that is connected to the rectifier 2884. The low pass filter and rectifier 2884 provides a rectified signal that is smoothed by the low pass filter. The data interpretation system 2826 has a comparator 2886, such as a threshold comparator, connected to the low pass filter and the rectifier 2884. The smoothed rectified signal is then squared by the comparator 2886.

Although the present invention has been described herein with reference to particular embodiments, it will be understood that this description is exemplary in nature and is not considered as a limitation on the scope of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

What is claimed and desired to be secured by Letters Patent is:

1. A sensing apparatus for measuring quantities convertible from changes in physical observations, said apparatus comprising:
a resonant structure responsive to said changes in said physical observations, said resonant structure including a magnetized element;
an electromagnetic coil operationally coupled to said magnetized element, said electromagnetic coil being an excitation coil magnetically coupled to said magnetized element to excite a resonance of said resonant structure; and,
a signal processor for processing movement of said resonant structure, said signal processor correlating said movement with regard to said changes in said physical observations so as to produce sensed data.

2. The apparatus as claimed in claim 1 wherein said changes in physical observations are changes in mechanical stress.

3. The apparatus as claimed in claim 1 wherein said changes in physical observations are changes in mass.

4. The apparatus as claimed in claim 1 wherein said sensed data includes physiological changes within a human body.

5. The apparatus as claimed in claim 4 wherein said physiological changes include changes in intraocular pressure.

6. The apparatus as claimed in claim 2 wherein said sensed data includes measurable physical occurrences selected from a group consisting of pressure changes, temperature changes, flow changes, rotation changes, acceleration changes, and sound changes.

7. The apparatus as claimed in claim 3 wherein said sensed data includes a measurable physical occurrence indicative of a presence of a chemical substance.

8. The apparatus as claimed in claim 2 wherein said resonant structure includes an adsorption mechanism that adsorbs a chemical substance such that said changes in physical observations is correlated to adsorption of said chemical substance by said adsorption mechanism.

9. The apparatus as claimed in claim 1 wherein said resonant structure resides within a vacuum environment so as to minimize damping losses.

10. The apparatus as claimed in claim 1 wherein said signal processor operates within a resonant sensing mode that is angular.

11. The apparatus as claimed in claim 1 wherein said signal processor operates within a resonant sensing mode that is linear.

12. The apparatus as claimed in claim 1 wherein said electromagnetic coil is also a pickup coil magnetically coupled to said magnetized element to sense a resonance of said resonant structure and to provide said resonance to said signal processor.

13. The apparatus as claimed in claim 1 wherein said electromagnetic coil is alternatively activated by circuitry within said signal processor to selectively form both said excitation coil and a pickup coil magnetically coupled to said magnetized element to sense said resonance of said resonant structure and to provide said resonance to said signal processor.

14. The apparatus as claimed in claim 1 wherein said resonant structure includes:
a substrate locatable in an environment to be monitored,
a flexible diaphragm hermetically sealed to said substrate and in communication with said environment to be monitored,
a sealed chamber encompassed by said substrate and said at least one flexible diaphragm, and
a resonant beam connected to said magnetized element, said resonant beam suspended within said sealed chamber and mechanically coupled to said flexible diaphragm,
wherein said magnetized element oscillates said resonant beam in response to an electromagnetic signal generated by said signal processor and formed by said electromagnetic coil.

15. The apparatus as claimed in claim 14 wherein said electromagnetic coil and said signal processor are locatable external to said environment to be monitored.

16. The apparatus as claimed in claim 15 wherein said environment to be monitored is intracorporeal, said substrate is attachable to a physiological structure, and said flexible diaphragm is capable of communication with a physiological fluid.

17. The apparatus as claimed in claim 16 wherein said substrate is attachable to a prosthetic device.

18. The apparatus as claimed in claim 16 wherein said environment to be monitored is an intraocular environment and said sensed data is intraocular pressure.

19. The apparatus as claimed in claim 17 wherein said environment to be monitored is an intraocular environment, said sensed data is intraocular pressure, and said prosthetic device is an intraocular lens.

20. The apparatus as claimed in claim 14 wherein said resonant beam is manufactured by photolithography and etching.

21. The apparatus as claimed in claim 14 wherein said substrate is formed from single crystal silicon.

22. The apparatus as claimed in claim 14 wherein said resonant beam is a polysilicon beam mounted to said substrate by at least one end of said polysilicon beam and spaced from said substrate between said at least once end and an opposite end of said polysilicon beam so as to allow free vibration of said polysilicon beam.

23. The apparatus as claimed in claim 22 wherein said polysilicon beam is formed from substantially undoped polysilicon treated to exhibit reduced tensile strain.

24. The apparatus as claimed in claim 14 wherein said flexible diaphragm is formed from polysilicon and surrounds said resonant beam, said flexible diaphragm being affixed to said substrate to define a primary cavity enclosing said resonant beam, said primary cavity being sealed off from surrounding atmosphere, and wherein an interior of said primary cavity is substantially evacuated.

25. The apparatus as claimed in claim 24 wherein said flexible diaphragm includes peripheral portions bonded to said substrate with channels extending through said peripheral portions from said primary cavity to a perimeter of said flexible diaphragm, said flexible diaphragm formed from material selected from a group consisting of silicon dioxide, polysilicon, silicon nitride, and combinations thereof, said material being formed within said channels and sealing off said channels such that atmospheric gases are prevented from entering or exiting said primary cavity through said channels.

26. The apparatus as claimed in claim 14 wherein said substrate further includes a displacement cavity, said displacement cavity sized such that a total internal cavity volume varies minimally with deflection of said flexible diaphragm over an operational range of displacement of said flexible diaphragm.

27. The apparatus as claimed in claim 14 wherein said resonant beam is suspended by said flexible diaphragm at one or more points thereupon such that said resonant beam is suspended beneath said flexible diaphragm.

28. The apparatus as claimed in claim 24 further including a depression in said substrate forming said primary cavity, wherein said resonant beam is attached to said flexible diaphragm in at least one point and to said substrate in at least another point.

29. The apparatus as claimed in claim 24 wherein said resonant beam is attached to said flexible diaphragm in at least two points such that said resonant beam is suspended entirely by said flexible diaphragm.

30. The apparatus as claimed in claim 14 wherein said resonant beam includes a stress-sensitive coating affixed thereon for varying stiffness of said resonant beam such that said resonant beam exhibits a variable resonant amplitude.

31. The apparatus as claimed in claim 14 wherein said resonant beam forms a structure selected from a group consisting of a bridge, a double ended tuning fork (DEFT), a cantilever, and a diaphragm.

32. The apparatus as claimed in claim 14 wherein said resonant beam is dynamically balanced.

33. The apparatus as claimed in claim 14 wherein said resonant beam exhibits mechanical amplification.

34. The apparatus as claimed in claim 14 wherein said resonant beam includes two resonant structures that are each used in a differential mode.

35. The apparatus as claimed in claim 14 wherein said magnetized element is formed from a permanent magnet.

36. The apparatus as claimed in claim 14 wherein said magnetized element is formed from a soft magnetic material.

37. The apparatus as claimed in claim 14 wherein said magnetized element is electroplated onto said resonant beam.

38. The apparatus as claimed in claim 14 wherein said magnetized element is formed from a conductor loop that exhibits a magnetic field in response to said electromagnetic signal.

39. The apparatus as claimed in claim 14 wherein said signal processor includes at least one gated receiver.

40. The apparatus as claimed in claim 14 wherein said signal processor forms at least one pulsed drive signal.

41. The apparatus as claimed in claim 14 wherein said signal processor is a grid dip meter.

42. The apparatus as claimed in claim 14 wherein motion of said resonant beam is detected optically.

43. The apparatus as claimed in claim 14 wherein motion of said resonant beam is detected acoustically.

44. The apparatus as claimed in claim 14 wherein motion of said resonant beam is detected electromagnetically by way of said electromagnetic coil in operational coupling with said signal processor.

45. A method of sensing physical observations within an environment, said method comprising:
operatively arranging a resonant structure in said environment and in proximity to a direct current bias field, said resonant structure including a magnetized element and being responsive to changes in said physical observations;
applying a magnetic field by way of an electromagnetic coil operationally coupled to said magnetized element;
measuring a plurality of successive values for magnetic resonance intensity of said resonant structure with a signal processor operating over a range of successive interrogation frequencies to identify a resonant frequency value of said resonant structure; and
using said resonant frequency value to identify sensed data correlating to said physical observation of said environment.

46. The method as claimed in claim 45 wherein said magnetic field is a time-varying magnetic field.

47. The method as claimed in claim 45 wherein said magnetic field is a magnetic field pulse.

48. The method as claimed in claim 45 wherein said magnetic field is a series of magnetic field pulses.

49. The method as claimed in claim 45 wherein said electromagnetic coil is an excitation coil magnetically coupled to said magnetized element to excite a resonance of said resonant structure.

50. The method as claimed in claim 49 wherein said signal processor processes movement of said resonant structure and correlates said movement with regard to said changes in said physical observations so as to produce said sensed data.

51. The method as claimed in claim 45 further including a step of detecting a transitory time-response of frequency emission intensity of said resonant structure with a receiver to identify a resonant frequency value of said resonant structure to be used for determining said sensed data.

52. The method as claimed in claim 51 further including a step of converting said detected transitory time-response into a frequency domain format so as to enable performance of a Fourier transform on said transitory time-response of magnetic vibration intensity detected.

53. The method as claimed in claim 45 further including steps of
providing a magnetic circuit exterior to said environment, and
concentrating magnetic flux in a region near said resonant structure so as to increase signal detection by said signal processor.
